(19) United States
(12) Patent Application Publication
(10) Pub. No.: US 2015/0210917 A1
(43) Pub. Date: Jul. 30, 2015

(54) ALKYLTHIOPHENE-RICH COMPOSITIONS, USES THEREOF AND METHODS OF MANUFACTURING THE SAME

(71) Applicant: GENIE IP B.V., Amsterdam (NL)

(72) Inventors: Harold Vinegar, Bellaire, TX (US); Scott Nguyen, Austin, TX (US)

(73) Assignee: GENIE IP B.V., Amsterdam (NL)

(21) Appl. No.: 14/412,699

(22) PCT Filed: Jul. 4, 2013
(86) PCT No.: PCT/IB2013/055491

§ 371 Date: Jan. 5, 2015

Related U.S. Application Data
(63) Continuation-in-part of application No. PCT/IB2013/053027, filed on Apr. 16, 2013.
Application No. PCT/IB2013/053027 is a continuation-in-part of application No. PCT/IB2013/054109, filed on May 19, 2013.
Application No. PCT/IB2013/054109 is a continuation-in-part of application No. PCT/US13/36674, filed on Apr. 15, 2013.
(60) Provisional application No. 61/668,003, filed on Jul. 4, 2012.
(60) Provisional application No. 61/738,393, filed on Dec. 17, 2012.
(60) Provisional application No. 61/759,309, filed on Jan. 31, 2013.
(60) Provisional application No. 61/787,183, filed on Mar. 15, 2013.

(51) Int. Cl. C09K 8/58; C10G 67/02; E21B 43/40; C07D 333/10; E21B 43/16; E21B 43/24

(52) U.S. Cl. CPC C09K 8/58; E21B 43/16; E21B 43/24; E21B 43/40; C07D 333/10; C10G 67/02

(57) ABSTRACT

Embodiments of the present invention relates to a pyrolysis-derived thiophenic composition having a high concentration of C1 and/or C2 and/or C3 alkylthiophenes. Preferably, the composition is derived from pyrolysis (e.g. by slow, low-temperature pyrolysis) of type IIs kerogen (e.g. of a kerogenous chalk). In some embodiments, the thiophenic composition may be used as an enhanced oil recovery (EOR) fluid. Some advantages of the presently-disclosed alkylthiophene-rich enhanced oil recovery (EOR) fluids are that (i) the alkyl-thiophene fluids have excellent solvency for heavy hydrocarbons, (ii) alkyl-thiophene fluids are insoluble in water; (iii) it is possible to blend the alkyl-thiophene fluids to a density of about 1.0 g/cc which matches extra heavy oils and bitumens and water; (iv) a boiling point of alkyl-thiophenes exceeds that of water, making it possible to inject heated EOR fluid and create steam in situ for steam distillation. Methods of use of the EOR fluid are disclosed herein.

### FIELD OF THE INVENTION

The present application relates to alkylthiophene-rich enhanced oil recovery (EOR) fluids and methods of manufacturing (e.g. by slow, low-temperature pyrolysis of sulfur-rich type IIs kerogen) the same.

### BACKGROUND AND RELATED ART

As world-wide energy needs continue to grow, there is concern that demand for energy may outstrip its supply. Technologies for improving the efficiencies of petroleum production become increasingly valuable.

Oil extraction from hydrocarbon reservoirs presently takes place in stages. Typically, the initial stage, known as primary recovery, involves drilling a well from the surface to a subsurface reservoir, where oil is trapped under pressure. A subsurface oil reservoir is understood to be an underground pool of a liquid mix of hydrocarbons that is contained within a geological formation beneath the surface of the earth. The subsurface reservoir may be penetrated by one or more wells, perforations that contact the subsurface reservoir and permit the removal of the liquid and gas hydrocarbons resident therein. When an oil reservoir containing oil under pressure is tapped by a drill hole, the reservoir's pressure forces its contents through the drill hole to the surface for collection. This process may continue until the pressure within the reservoir is no longer sufficient to expel the oil contained therein. When the pressure in the reservoir is depleted but there is still oil available, artificial lifts (such as pumps) may be used to bring the oil to the surface.

The wells used for removing the contents of the reservoir may also be used for injecting substances into the reservoir to enhance the extraction of its contents. For example, such materials as water, brine, steam, and mobilization chemicals such as surfactants may be injected. A well from which oil is recovered is known as a production well. A well through which substances are injected is known as an injection well.

Injection techniques are particularly useful when the pressure within the reservoir decreases so that supplemental measures are useful to increase the recovery of oil contained within the reservoir. Techniques used under these circumstances may be termed secondary recovery techniques. For example, the pressure within the reservoir may be increased by injecting water, steam or gas into the reservoir. Injecting water into a well to increase recovery of oil is called “waterflood.” The combination of primary and secondary oil recovery only removes a certain amount of the total oil content from an oil reservoir, approximately between 20% and 60%.

Hence, a large amount of the original oil remains in the reservoir after secondary recovery techniques. In large oil fields, over a billion barrels of oil may remain after secondary recovery efforts. The percentage of unrecovered hydrocarbons is largest in oil fields with complex lithologies, and the petroleum fractions left behind tend to be the heavier hydrocarbon materials and those liquid materials that may be trapped by high capillary forces in the micron-sized pores in the reservoir rock or adsorbed onto mineral surfaces through irreducible oil saturation. There may also be pools of bypassed oil within the rock formations surrounding the main reservoir. Retrieving the normally immobile oil residing in the oil field after primary and secondary recovery is referred to herein as “tertiary recovery” or “enhanced oil recovery” (EOR).

Current EOR techniques may be able to remove an additional 5% to 20% of the oil remaining in a reservoir. Techniques currently available leave significant amounts of oil behind. Such techniques may also be expensive to carry out and inefficient. For example, gelled or crosslinked water-soluble polymers may be introduced that alter the permeability of geological formations to make waterflooding more effective. Polymers, either preformed or gelled/crosslinked in situ, may be introduced into the reservoir from external sources. Polymer-based techniques are costly processes, though.

Other recovery techniques may include flooding with polymers, alkali, or other chemical solutions, and various thermal processes. Alternatively, gases such as carbon dioxide, miscible gas or nitrogen may be injected into the reservoir, where they expand and push additional oil out through the production wellbores, and where they may affect the viscosity of the remaining oil, thereby improving its flow rate on egress.

As another example, EOR may take place using a variety of externally-introduced chemical agents that may be used to increase the efficacy of waterflooding. These agents fall into two categories. One type of chemical agent may be a surfactant material that can alter the surface tension that adheres oil, water and rock together within the formation. The second type of chemical agent is viscous enough to slow the passage of water through the rock matrix so that the trapped oil can be pushed out more effectively. Chemical techniques for EOR may also be disadvantageous. Existing surfactants, for example, may adversely affect properties of oil-bearing rock formations and thereby damage reservoirs. Also, these surfactants, being of low viscosity, may not be effective in pushing the oil out of the pores where it is trapped. In addition, these surfactants may not be able to function effectively under the high temperature and high pressure conditions where they are used. Certain surfactants, such as petroleum sulfonates or their derivatives, are also particularly difficult to remove from the desired petroleum once it has been extracted. As an additional problem, surfactants are typically used with waterflooding techniques, leading to the production of highly stable emulsions containing mostly water with very little oil. In sum, with existing surfactant techniques, it is difficult to extract oil from rock and difficult to remove it from the water used to flush it out of the reservoir. The costs associated with these processes and their technical limitations have limited the widespread adaptation of these EOR techniques.

Many variations on the aforesaid systems and methods have been proposed. For example, U.S. Patent Appl. No. 20070079964 discloses the use of aliphatic anionic surfactants. U.S. Patent Appl. No. 20060046948 discloses the use of alkyl polyglycosides. U.S. Pat. No. 6,225,263 discloses the use of alkylglycol ethers. U.S. Pat. No. 6,475,290 discloses the use of lignin sulfonates. U.S. Pat. No. 5,911,276 discloses the use of lignin U.S. Pat. No. 4,790,382 discloses the use of alkylated, oxidized lignin

In addition to petroleum reservoirs as described above, petroleum may be extracted from formations called oil sands or tar sands. Oil sands, also called tar sands, are mixtures of sand or clay, water and extremely heavy crude oil (e.g., bitumen). For example, a major formation of oil sands in Alberta, Canada, contains material that is approximately 90% sand, 10% crude oil, and water. Oil sand formations are understood to comprise naturally-occurring petroleum deposits in which the lighter fractions of the oil have been lost, and the remaining heavy fractions have been partially degraded by bacteria. The crude oil is extra heavy crude and can be characterized as a naturally occurring viscous mixture of hydrocarbons that are generally heavier than pentane. The petroleum contained in these formations is a viscous, tar-like substance that is admixed with clay, sand and other inorganic particulate matter. Accordingly, it is harder to refine and generally of lesser quality than other crudes. While there is great variability, depending on the oil sands source, the mineral matter in oil sands typically includes a fairly uniform white quartz sand, silt, clay, water, bitumen and other trace minerals, such as zirconium, pyrite and titanium. The bitumen content of oil sands may be as high as 18%, or it can be substantially lower.

As described above, conventional crude oil in reservoirs may be readily extracted by drilling wells into the formation, because the light or medium density oil in such reservoirs can flow freely out. By contrast, there is no free-flowing oil in an oil sand formation. Instead, these deposits must be strip mined or their petroleum content must be heated in situ until it flows.

In the strip mining method, oil sands are dug up from a surface mine and are transported and washed to remove the oil Mining methods typically involve a number of steps, beginning with excavation and ore size reduction, followed by slurry formation with water and sodium hydroxide. The slurry is then treated with flotation agents (typically kerosene), frothing agents (methylisobutyl carbinol is common), and air is passed through the slurry to create a bitumen froth. This mixture is transported through several kilometers of pipeline, creating a mechanical as well as chemical separation of the bitumen from the inorganic sand and silt. The pipeline leads to a separation tank that allows the froth to be skimmed off while the inorganic material falls to the bottom. Since the bitumen is much more viscous than standard crude oil, it must be either mixed with a lighter petroleum or chemically processed so that it is flowable enough for transport. Further processing removes water and solids, following which the bitumen may be processed to form synthetic crude oil. Using this method, about two tons of tar sands produce one barrel of oil.

Much of the oil sands reserve is located deep below the surface, so the strip mining technique is not applicable. For these formations, a variety of in situ methods are available to extract bitumen from underground formations via specialized drilling and extraction techniques. These methods typically use a great amount of energy in the form of steam to heat the trapped bitumen. The heated bitumen has a lower viscosity and can then flow, slowly, to a production well. The steam-softened bitumen may form an emulsion with the water from the steam and drain to a wellhead within the formation from which it is pumped to the surface.

U.S. Patent Application Publication Number 2006/0254769 discloses a system including a mechanism for recovering oil and/or gas from an underground formation, the oil and/or gas comprising one or more sulfur compounds; a mechanism for converting at least a portion of the sulfur compounds from the recovered oil and/or gas into a carbon disulfide formulation; and a mechanism for releasing at least a portion of the carbon disulfide formulation into a formation.

In US 20100307759 it was proposed to inject a miscible carbon disulfide enhanced oil recovery formulation into a formation via a first array of wells while the second array of wells comprises a mechanism to produce oil and/or gas. There are a number of problems associated with using carbon disulfide as an EOR fluid for heavy oil formations. For example, due to its low atmospheric boiling point of around 50 degrees Celsius, it can only be heated to a limited extent upon injection if it is to remain in the liquid phase. A density of carbon disulfide is about 1.25 g/cc and thus is gravitationally unstable relative to even heavy oils. It has a water solubility of 0.22% @22 degrees Celsius.

### SUMMARY OF EMBODIMENTS

Embodiments of the present invention relate to alkyl-thiophene compositions, uses thereof, and methods of manufacturing the same. In one particular embodiment, it is possible to manufacture, from hydrocarbon pyrolysis liquids derived from slow, low-temperature pyrolysis of type IIs kerogen at least of: (i) a highly concentrated C2-alkylthiophene composition; (ii) a highly concentrated C3-alkylthiophene composition; and (iii) a highly concentrated C2-C3 alkylthiophene composition. By separating (e.g. by fractional distillation) C2-alkylthiophenes and/or C3-alkylthiophenes from the hydrocarbon pyrolysis liquids, it is possible to (i) reduce the need for and cost of hydrotreating (i.e. when deriving transportation fuel from the hydrocarbon pyrolysis liquids) since at least some sulfur heterocyclic compounds are separated out and recovered without being hydrotreated; and (ii) instead, derive a valuable product from the type IIs-kerogen-derived hydrocarbon pyrolysis liquids.

In a preferred embodiment, the ‘derived product’ is an enhanced oil recovery (EOR) fluid. Nevertheless, this is not a limitation and many other uses (e.g. as a cleaning agent, as a solvent, as a feedstock for agrochemicals or pharmaceuticals, or any other use) are contemplated.

In some embodiments, the present inventors are now disclosing, for the first time, the use of alkyl-thiophenes as an enhanced oil recovery (EOR) fluid. Furthermore, the present inventors are now disclosing a novel process for economically synthesizing fluids rich in alkyl-thiophenes by recovering primarily light-molecular-weight alkyl-thiophene compounds from oil or from pyrolysis fluids derived from sulfur-rich type IIs kerogen.

In different embodiments, advantages of the presently-disclosed EOR fluids may include any of the following: (i) the alkyl-thiophene fluids have excellent solvency for heavy hydrocarbons, including asphaltenes and resins—unlike propane which is used as a deasphalter; (ii) alkyl-thiophene fluids are insoluble in water; (iii) it is possible to blend the alkyl-thiophene fluids to a density of about 1.0 g/cc which matches extra heavy oils and bitumens and water; (iv) a boiling point of alkyl-thiophenes exceeds that of water, making it possible to inject heated EOR fluid and create steam in situ for steam distillation; (v) when using alkyl-thiophene as an EOR fluid in a bitumen-rich formation (e.g. a tar-sands formation), it is possible to separate the alkyl-thiophenes from the produced bitumen by atmospheric distillation in a narrow temperature range, optionally followed by extractive distillation with a polar solvent like NMP (N-methyl-2-pyrrolidinone); (vii) alkyl-thiophene rich fluids are stable at high temperatures; (viii) as a result of the presently-disclosed manufacturing technique relating to type IIs kerogen, alkyl-thiophene fluids are relatively inexpensive and available in large quantities; (viii) alkyl-thiophene-rich fluids are recoverable at high efficiency by waterflooding (ix) any EOR fluid remaining underground results in sequestering of sulfur, thereby lowering sulfur emissions.

Experimental data related to pyrolysis liquids formed by slow, low-temperature heating of type IIs-kerogen has indicated that a majority of sulfur compounds within the pyrolysis liquids derived from the type IIs kerogen are alkyl-thiophenes.

In particular, as a result of the slow and/or low-temperature pyrolysis, the hydrocarbon pyrolysis liquids derived therefrom are rich in C1-C3 alkyl-thiophenes—specifically, methyl-thiophenes, di-methyl-thiophenes and tri-methyl-thiophenes.

In order to form the alkyl-thiophene-rich EOR fluid from the low-temperature-pyrolysis derived hydrocarbon formation fluids, these fluids may be processed to form a mixture comprising relatively high concentrations of C1alkylthiophenes and/or C2 alkylthiophenes and/or C3 alkylthiophenes. This may be carried out by fractional distillation of the pyrolysis formation fluids. In one example, the fractional distillation yields a mixture of C1-C3 alkylthiophenes together with hydrocarbons C_{N}H_{M} hydrocarbons (N and M are both positive integers) having a boiling point that substantially matches the C1, C2 and/or C3 alkylthiophenes.

To further concentrate the C1, C2 and/or C3 alkylthiophenes, the C_{N}H_{M} hydrocarbons may be separated out—for example, by an an extractive distillation with a polar solvent like NMP (N-methyl-2-pyrrolidinone). Alternately or additionally, the C_{N}H_{M} hydrocarbons may be separated out by a cryogenic separation.

In some embodiments, an EOR fluid is formed from the alkyl-thiophene rich composition.

The EOR fluid may be injected into a hydrocarbon-containing subsurface formation—for example, a tar-sands formation or a heavy-oil formation. This forces hydrocarbon fluids (e.g. having a relatively high viscosity or density) within the formation to enter production wells, to facilitate production from the formation. The EOR fluid may be heated and used in a huff-and-puff cyclic injection. The EOR fluid may also be used as a miscible displacement fluid that is gravity-matched to the heavy oil and brine in the reservoir.

In some embodiments, the produced hydrocarbon fluids are recovered as part of a mixture together with alkyl-thiophenes of the EOR fluid. It is possible, once again, to employ fractional distillation to separate the alkyl-thiophenes from the produced hydrocarbon fluids. This obviates the need to hydrotreat the alkyl-thiophenes from the produced hydrocarbon fluids, and allows their re-use.

In some embodiments, an oil recovery method comprises: injecting an enhanced oil recovery (EOR) fluid comprising methyl-thiophenes and/or di-methyl-thiophenes and/or tri-methyl thiophenes into a target subsurface hydrocarbon-containing formation via one or more wells situated therein; and recovering, via one or more wells in the target formation, a fluid comprising oil and/or bitumen of the target formation. In some embodiments, the produced fluid and the injected methyl-thiophenes and/or di-methyl-thiophenes of the EOR fluid.

In some embodiments, a majority or a substantial majority (i.e. at least 75% wt/wt) of the alkylthiophenes of the EOR fluid are C1-C3 alkylthiophenes. In some embodiments, a majority or a substantial majority (i.e. at least 75% wt/wt) of the alkylthiophenes of the EOR fluid are methyl-thiophenes and/or di-methyl-thiophenes and/or tri-methyl thiophenes.

In some embodiments, the EOR fluid is at least 50% wt/wt or at least 75% wt/wt thiophenes.

In some embodiments, the EOR fluid is at least 15% wt/wt or at least 20% wt/wt sulfur.

The oil and/or bitumen may be recovered via the same well through which the EOR fluid was injected, or from a different well.

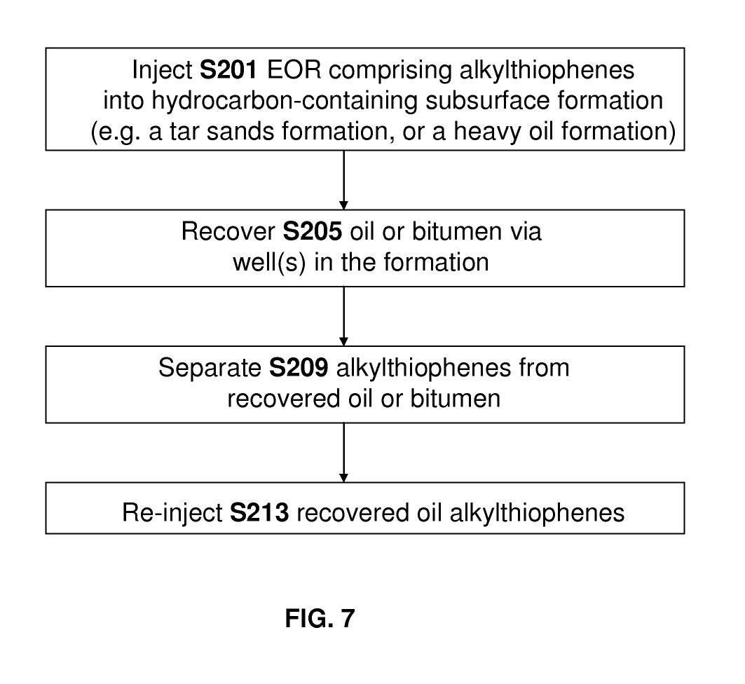

The method may be practiced as a “huff-n-puff” or cyclical injection and production method—for example, see FIGS. 7A-7B. There may be one cycle of injection and production, two cycles, or N cycles, where N may be at least 5, or at least 10, or at least 15, or at least 20, or more cycles of injection and production.

The hydrocarbon-bearing formation may contain bitumen or heavy oil. alkylthiophenes (e.g. C1-C4 or C1-C3 or C2-C3 alkylthiophenes) are particularly good solvents for heavy oils and bitumens. Unlike hot propane injection, which may de-asphalt the bitumen because the asphaltenes are not soluble in the propane, methyl thiophenes and dimethyl thiophenes are excellent solvents for asphaltenes and resins that comprise much of the compounds in heavy oil and bitumen.

In some embodiments, the EOR fluid acts as a solvent for heavy oil and/or bitumen contained in the formation. Moreover, the methyl thiophenes and dimethyl thiophenes may have about the same density as the heavy oil or bitumen. For example, the density of a bitumen may be about 1.0 g/cc, closely density-matched to methyl, dimethyl and trimethyl thiophenes which respectively have densities of about 1.01 g/cc, about 0.994 g/cc and about 0.98 g/cc)—it is thus possible to density-match the EOR fluid to the exact heavy oil or bitumen density. This prevents gravitational override or underride during injection of the miscible solvent into the formation.

An additional highly desirable and environmentally-friendly feature of methyl, dimethyl and trimethyl thiophenes is that they are not water soluble. This is in contrast to many of the aromatic hydrocarbons like benzene and toluene which have slight water solubilities. Thus there is no mixing with water if a spill should occur on the surface, and generally no risk to aquifers from injection into the hydrocarbon reservoir.

Another desirable feature is that the methyl thiophenes and di methyl thiophenes are stable to high temperatures. Thus they may be heated at the surface and injected at elevated temperatures into the subsurface hydrocarbon-bearing formation. The invention includes heating and injecting the EOR fluid at a temperature above the boiling point of the in situ brine. Thus when the water boils in the pore spaces, the steam distills and removes oil and/or bitumen contained in the formation.

In some embodiments, when mixed as a solvent with bitumen of the subsurface formation and within the subsurface formation, the EOR fluid lowers the viscosity of the bitumen by a factor of at least 10, preferably of at least 100.

In some embodiments, the injected EOR fluid is pre-heated to a temperature of between 50 degrees Celsius and 200 degrees Celsius, and when mixed as a hot solvent with bitumen within the subsurface formation, at a temperature of between 50 degrees Celsius and 200 degrees Celsius, the EOR fluid lowers the viscosity of the bitumen by a factor of at least 100, 1,000, and preferably by at least 10,000.

Some embodiments relate to a method of producing a mixture of EOR fluid, oils and/or bitumen from the production well, plus distilling the EOR fluid from the recovered oil and/or bitumen, and reinjecting the EOR fluid into the target formation for additional recovery of oil and/or bitumen.

The reinjection may occur as huff-and-puff cyclic injection or as a drive from multiple wells in a well pattern.

Some of the EOR fluid remaining in the formation after the oil and/or bitumen is recovered may be removed later by brine injection. The brine is almost perfectly matched in density to the EOR fluid, and because the brine is higher in viscosity, the waterflood is stable and able to recover a large percentage of the EOR fluid remaining in the reservoir.

Residual EOR fluid after waterflood can be safely stored in the reservoir because it is chemically stable, it is insoluble in water and contains a large amount of sulfur. There are no known environmental risks associated with storing methyl thiophenes or di methyl thiophenes in a depleted oil reservoir.

Some embodiments relate to a method for producing an EOR fluid from a high sulfur oil derived from a Type IIs kerogen. The method may include separation in a set of fractionating column(s) based on boiling point, a chemical extraction step using a polar organic solvent like NMP and/or cryogenic separation of thiophenic compounds from aromatic hydrocarbons.

It is now disclosed a thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) at least a majority or at least a substantial majority or substantially all of the alkylthiophenes of the composition are derived from pyrolysis of type IIs kerogen.

It is now disclosed a thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) a δ³⁴S(‰) value of the composition is at least +0.75 or at least +1.0 or at least +1.25 or at least +1.5, the δ³⁴S(‰) value describing deviations from the V-CFT (Vienna Canyon Diablo Troilite) standard.

In some embodiments, K=2 and L=3.

It is now disclosed a thiophenic composition comprising at least 50% wt/wt or at least 70% wt/wt or at least 95% wt/wt or at least 99% wt/wt CL alkylthiophenes, wherein L is a positive integer equal to at most 3, and at least a majority or at least a substantial majority or substantially all of the alkylthiophenes of the mixture are derived from pyrolysis of type IIs kerogen.

It is now disclosed a thiophenic composition comprising at least 50% wt/wt or at least 70% wt/wt or at least 95% wt/wt or at least 99% wt/wt CL alkylthiophenes, wherein L is a positive integer equal to at most 3, and a δ³⁴S(‰) value of the composition is at least +0.75 or at least +1.0 or at least +1.25 or at least +1.5, the δ³⁴S(‰) value describing deviations from the V-CFT (Vienna Canyon Diablo Troilite) standard.

In some embodiments, L=1 or L=2 or L=3.

In some embodiments, the composition comprises at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

It is now disclosed a thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) the composition comprises at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

In some embodiments, K=2 and L=3.

It is now disclosed a thiophenic composition comprising at least 50% wt/wt or at least 70% wt/wt or at least 95% wt/wt or at least 99% wt/wt CL alkylthiophenes, wherein L is a positive integer equal to at most 3, and the composition comprises at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

In some embodiments, L=1 or L=2 or L=3.

In some embodiments, the polar organic solvent is capable of selectively extracting methylthiophenes, dimethylthiophenes and trimethylthiophenes from a liquid mixture involving liquid-phase C_{N}H_{M} hydrocarbon compounds.

In some embodiments, a boiling point of the organic solvent is at least 180 degrees Celsius or at least 190 degrees Celsius.

In some embodiments, the organic solvent is NMP.

In some embodiments, the organic solvent is immiscible with water.

In some embodiments, the composition comprises at least 0.1% wt/wt or at least 0.3% wt/wt or at least 0.5% wt/wt or at least 1% wt/wt or at least 3% wt/wt or at least 5% wt/wt or at least 10% wt/wt C_{N}H_{M} hydrocarbon compounds, wherein an individual-compound atmospheric boiling point of each C_{N}H_{M} hydrocarbon compound is between about 80° C. and about 175° C.

It is now disclosed a composition comprising at least 50% wt/wt or at least 70% wt/wt or at least 95% wt/wt or at least 99% wt/wt CL alkylthiophenes, wherein L is a positive integer equal to at most 3, and the composition comprises at least 0.1% wt/wt or at least 0.3% wt/wt or at least 0.5% wt/wt or at least 1% wt/wt or at least 3% wt/wt or at least 5% wt/wt or at least 10% wt/wt C_{N}H_{M} hydrocarbon compounds.

In some embodiments, the individual-compound atmospheric boiling point of each C_{N}H_{M} hydrocarbon compound is at least 110° C. or at least 135° C. or at least 155° C.

In some embodiments, the individual-compound atmospheric boiling point of each C_{N}H_{M} hydrocarbon compound (i) matches that of methyl-thiophene, dimethyl-thiophene and tri-methyl-thiophene and/or (ii) has a value between about 113° C. and about 117° C. or between 139° C. and about 141° C. or between about 161° C. and about 163° C.

In some embodiments, the composition comprises at least 50% wt/wt or at least 70% wt/wt or at least 95% wt/wt or at least 99% wt/wt methylthiophene.

In some embodiments, the composition comprises at least 50% wt/wt or at least 70% wt/wt or at least 95% wt/wt or at least 99% wt/wt dimethylthiophene.

In some embodiments, the composition comprises at least 50% wt/wt or at least 70% wt/wt or at least 95% wt/wt or at least 99% wt/wt trimethylthiophene.

In some embodiments, the composition comprises at least 99% wt/wt dimethylthiophene.

In some embodiments, the composition is derived from pyrolysis of type IIs kerogen of a kerogeneous chalk

In some embodiments, the composition is derived from pyrolysis of type IIs kerogen of a Ghareb formation kerogeneous chalk.

In some embodiments, the composition comprises at least 10 PPM or at least 25 PPM or at least 50 PPM or at least 100 PPM or at least 0.25% wt/wt olefins. In some embodiments, the composition comprises at least 10 PPM silicon.

It is now disclosed a method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 139 degrees Celsius and 141 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

It is now disclosed a method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 160 degrees Celsius and 165 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

It is now disclosed a method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 115 degrees Celsius and 118 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

It is now disclosed a method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 115 degrees Celsius and at most 175 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C1-C3 alkylthiophenes.

It is now disclosed a method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 135 degrees Celsius and at most 175 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C2-C3 alkylthiophenes.

It is now disclosed a method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 139 degrees Celsius and at most 141 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C2 alkylthiophenes.

It is now disclosed a method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 161 degrees Celsius and at most 163 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C3 alkylthiophenes.

It is now disclosed an oil recovery method comprising:
a. injecting an enhanced oil recovery (EOR) fluid comprising alkylthiophenes into a target subsurface hydrocarbon-containing formation via one or more wells situated therein, a majority of sulfur compounds of the EOR fluid being alkylthiophenes; and
b. recovering, via one or more wells in the target formation, oil and/or bitumen and/or pyrolysis liquids and/or mobilized hydrocarbon liquids that are mobilized by the injected EOR fluid.

In some embodiments, a density of the injected EOR fluid is between 0.95 and 1.05 g/cc.

In some embodiments, the injected EOR fluid comprises primarily alkylthiophenes, or at least 75% wt/wt alkylthiophenes, or at least 90% wt/wt alkylthiophenes, or at least 95% wt/wt alkylthiophenes or at least 99% wt alkylthiophenes.

In some embodiments, an atmospheric boiling point of the EOR fluid is between about 135° C. and about 175° C.

In some embodiments, a majority, or a substantial majority, of alkylthiophenes of the injected EOR fluid are C1-C3 alkylthiophenes.

In some embodiments, a majority, or a substantial majority, of alkylthiophenes of the injected EOR fluid are methyl-thiophene or di-methyl-thiophene or tri-methyl-thiophene.

In some embodiments, a majority, or a substantial majority, of alkylthiophenes of the injected EOR fluid are C2-C3 alkylthiophenes.

In some embodiments, a majority, or a substantial majority, of alkylthiophenes of the injected EOR fluid are di-methyl-thiophene, or tri-methyl-thiophene.

In some embodiments, a majority, or a substantial majority, of alkylthiophenes of the injected EOR fluid are C2 alkylthiophenes.

In some embodiments, a majority, or a substantial majority, of alkylthiophenes of the injected EOR fluid are di-methyl-thiophenes.

In some embodiments, a majority, or a substantial majority, of alkylthiophenes of the injected EOR fluid are C3 alkylthiophenes.

In some embodiments, a majority, or a substantial majority, of alkylthiophenes of the injected EOR fluid are tri-methyl-thiophenes.

In some embodiments, a majority, or a substantial majority, of alkylthiophenes of the injected EOR fluid are methyl-thiophenes.

In some embodiments, the EOR fluid is insoluble in water.

In some embodiments, the hydrocarbon-containing formation is at residual hydrocarbon saturation following waterflood.

In some embodiments, a temperature of the injected EOR fluid is at least 100 degrees Celsius or at least 200 degrees Celsius.

In some embodiments, a majority of the recovered alkylthiophenes are re-injected into the formation or into another subsurface formation.

In some embodiments, further comprising distilling from the recovered hydrocarbon mixture a majority of the alkylthiophenes to form a second mixture.

In some embodiments, a majority of the second mixture is re-injected into target subsurface hydrocarbon-containing formation or injected into a different subsurface hydrocarbon-containing formation.

In some embodiments, the second mixture has an alkylthiophene concentration that is at most 50% that of the recovered hydrocarbon mixture.

In some embodiments, the injecting and the producing is via the same well.

In some embodiments, the injecting and the producing is via different wells.

In some embodiments, within the subsurface formation the EOR fluid acts as a solvent for oil and/or bitumen contained in the formation.

In some embodiments, within the subsurface formation the EOR fluid boils the in situ brine which steam distills, within the formation, oil and/or bitumen contained in the formation.

In some embodiments, when mixed with bitumen of the subsurface formation and within the subsurface formation, the EOR fluid lowers the viscosity of the bitumen by a factor of at least 10, preferably of at least 100.

In some embodiments, the injected EOR fluid is pre-heated to a temperature of between 50 degrees Celsius and 200 degrees Celsius.

In some embodiments, when mixed with bitumen of the subsurface formation and within the subsurface formation, at a temperature of between 50 degrees Celsius and 200 degrees Celsius, the EOR fluid lowers the viscosity of the bitumen by a factor of at least 100, and preferably by at least 1000.

In some embodiments, plus distilling the EOR fluid from the recovered oil and/or bitumen, re-injecting the EOR fluid into the target formation for additional recovery of oil and/or bitumen.

In some embodiments, the EOR fluid is at least 10% wt/wt or at least 15% wt/wt or at least 20% wt/wt sulfur.

In some embodiments, an atmospheric boiling point of the EOR fluid is between about 80° C. and about 175° C.

In some embodiments, an atmospheric boiling point of the EOR fluid is in one of the ranges: (i) between about 113 degrees Celsius and about 119 degrees Celsius; (ii) between about 137 degrees Celsius and about 143 degrees Celsius; and (iii) between about 159 degrees Celsius and about 165 degrees Celsius/

In some embodiments, the atmospheric boiling point of the EOR fluid is at least 100° C. or at least 110° C.

In some embodiments, a majority, or a substantial majority, of alkylthiophenes of the injected EOR fluid are thiophene C₄H₄S or C1-C4 alkylthiophenes.

In some embodiments, the target formation is a kerogenous chalk.

It is now disclosed a method of production of a thiophenic mixture, the method comprising:
a. pyrolyzing type IIs kerogen to generate condensable pyrolysis fluids therefrom; and
b. forming from the pyrolysis liquids a thiophenic fluid mixture comprising at least 50% wt/wt alkylthiophenes.

In some embodiments the thiophenic fluid mixture formed from the pyrolysis liquids comprises at least 50% wt/wt or at least 70% wt/wt or at least 95% wt/wt or at least 99% wt/wt methylthiophenes.

In some embodiments, the thiophenic fluid mixture formed from the pyrolysis liquids comprises at least 50% wt/wt or at least 70% wt/wt or at least 95% wt/wt or at least 99% wt/wt dimethylthiophenes.

In some embodiments, the thiophenic fluid mixture formed from the pyrolysis liquids comprises at least 50% wt/wt or at least 70% wt/wt or at least 95% wt/wt or at least 99% wt/wt trimethylthiophenes.

In some embodiments, the pyrolyzing is performed in situ.

In some embodiments, the pyrolysis occurs primarily at temperatures below 290 degrees Celsius.

In some embodiments, the forming includes (i) subjecting the pyrolysis liquids or a derivative thereof to a fractional distillation to recover a fraction having boiling points between at least 135 degrees Celsius and at most 175 degrees Celsius; and (ii) subjecting fluids of the recovered fraction to an extractive distillation with a polar organic solvent having a boiling point of at least 180 degrees Celsius.

In some embodiments, the thiophenic fluid mixture comprises at least 75% wt/wt alkylthiophenes, or at least 90% wt/wt alkylthiophenes, or at least 95% wt/wt alkylthiophenes or at least 99% wt alkylthiophenes.

In some embodiments, the thiophenic fluid mixture comprises at least 75% wt/wt methyl-thiophenes, or at least 90% wt/wt methyl-thiophenes, or at least 95% wt/wt methyl-thiophenes or at least 99% methyl-thiophenes.

In some embodiments, the thiophenic fluid mixture comprises at least 5% wt/wt or at least 10% wt/wt or at least 20% wt/wt hydrocarbons C_{N}H_{M} hydrocarbons wherein N and M are both positive integers, and a value of N is between 5 and 12.

In some embodiments, (i) the forming includes subjecting the condensable pyrolysis fluids or a derivative thereof to a distillation process to recover fluids having an atmospheric boiling point in the 75° C.-175° C. range and (ii) the thiophenic fluid mixture is derived from the 75° C.-175° C. range fluids recovered by the distillation.

In some embodiments, (i) the forming includes subjecting the condensable pyrolysis fluids or a derivative thereof to a distillation process to recover fluids having an atmospheric boiling point in the 135° C.-175° C. range and (ii) the thiophenic fluid mixture is derived from the 135° C.-175° C. range fluids recovered by the distillation.

In some embodiments, the forming includes subjecting the condensable pyrolysis fluids to a chemical extraction process by a polar organic solvent having a boiling point above 160 degrees Celsius or above 180 degrees Celsius.

In some embodiments, the forming includes subjecting the condensable pyrolysis fluids to a chemical extraction process by a polar organic solvent which differentiates between alkylthiophenes and C_{N}H_{M} hydrocarbons wherein N and M are both positive integers, and a value of N is between 5 and 12.

In some embodiments, the forming includes subjecting the condensable pyrolysis fluids to a cryogenic separation process.

In some embodiments, a majority, or a substantial majority, of alkylthiophenes of the thiophenic fluid mixture are C1-C3 alkylthiophenes.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—and any combination of features can be included in any embodiment and/or omitted from any embodiments.

Definitions

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

If two numbers A and B are “on the same order of magnitude”, then ratio between (i) a larger of A and B and (ii) a smaller of A and B is at most 15 or at most 10 or at most 5.

Unless specified otherwise, a ‘substantial majority’ refers to at least 75%. Unless specified otherwise, ‘substantially all’ refers to at least 90%. In some embodiments ‘substantially all’ refers to at least 95% or at least 99%.

Embodiments of the present invention relate to compositions (e.g. oils) containing one or more types of heterocyclic compounds including (i) sulfur heterocyclic compounds such as the single-ring alkylthiophenes, or the multi-ringed alkylbenzothiophenes or alkyldibenzothiophenes and (ii) nitrogen heterocyclic compounds such as the single-ringed alkylpyridines or alkylpyrroles, or the multi-ringed alkylquinolines, alkylisoquinolines, alkylacridines, and alkylindoles, and alkylcarbazoles.

The term ‘alkylthiophenes’ includes thiophene C₄H₄S as well as alkylated thiophenes. ‘Alkylated thiophenes’ are thiophenes where an alykl group is bonded to one or more locations on the thiophene ring. Thiophene C₄H₄S is an ‘alkylthiophene’ but is not an ‘alkylated thiophene.’ Examples of alkylated thiophenes include but are not limited to methyl thiophenes, di-methyl thiophenes, ethyl thiophenes, ethyl methyl thiophenes, propyl thiophenes, etc. Analogous definitions (i.e. analogous to ‘alkyl-thiophenes’) apply to the multi-ring sulfur heterocyclic compounds (i.e. alkylbenzothiophenes and alkyldibenzothiophenes), to the single-ring nitrogen heterocyclic compounds (i.e. alkylpyridines and alkylpyrroles), and to the multi-ring nitrogen heterocyclic compounds (i.e. alkylquinolines, alkylisoquinolines alkylacridines, and alkylindoles and alkylcarbazoles).

By way of example, methyl thiophenes are a ‘C1alkylthiophene’ because the total number of carbon atoms of alkyl groups bonded to a member of the thiophene ring is exactly 1. Both di-methyl thiophenes and ethyl thiophenes are ‘C2 alkylthiophenes’ because the total number of carbon atoms of bonded-alkyl group(s) bounded to a member of thiophene ring is exactly 2. C3 alkylthiophenes are molecules where the total number of carbon atoms of bonded-alkyl group(s) bounded to a member of thiophene ring is exactly 3—thus, C3 alkylthiophenes include tri-methyl thiophenes, methyl ethyl thiophenes and propyl thiophenes. Analogous definitions (i.e. analogous to ‘alkylthiophenes’) apply to the multi-ring sulfur heterocyclic compounds (i.e. alkylbenzothiophenes and alkyldibenzothiophenes), to the single-ring nitrogen heterocyclic compounds (i.e. alkylpyridines and alkylpyrroles), and to the multi-ring nitrogen heterocyclic compounds (i.e. alkylquinolines, alkylisoquinolines alkylacridines, and alkylindoles and alkylcarbazoles).

For a positive integer N, the terms ‘CN alkylthiophenes’ and ‘CN thiophenes’ are used synonymously and refer to alkylthiophenes (which also happen to be ‘alkylated thiophenes’) where the total number of carbon atoms of bonded-alkyl group(s) bounded to a member of thiophene ring is exactly N. Analogous definitions (i.e. analogous to ‘alkylthiophenes’) apply to the multi-ring sulfur heterocyclic compounds (i.e. alkylbenzothiophenes and alkyldibenzothiophenes), to the single-ring nitrogen heterocyclic compounds (i.e. alkylpyridines and alkylpyrroles), and to the multi-ring nitrogen heterocyclic compounds (i.e. alkylquinolines, alkylisoquinolines alkylacridines, and alkylindoles and alkylcarbazoles).

For a positive integer N, the terms ‘CN+alkylthiophenes’ and ‘CN+thiophenes’ are used synonymously and refer to alkylthiophenes (which also happen to be ‘alkylated thiophenes’) where the total number of carbon atoms of bonded-alkyl group(s) bounded to a member of thiophene ring is greater than or equal to N. Analogous definitions (i.e. analogous to ‘alkylthiophenes’) apply to the multi-ring sulfur heterocyclic compounds (i.e. alkylbenzothiophenes and alkyldibenzothiophenes), to the single-ring nitrogen heterocyclic compounds (i.e. alkylpyridines and alkylpyrroles), and to the multi-ring nitrogen heterocyclic compounds (i.e. alkylquinolines, alkylisoquinolines alkylacridines, and alkylindoles and alkylcarbazoles).

For positive integers N, M (M>N), the terms ‘CN-CM alkylthiophenes’ and ‘CN+thiophenes’ are used synonymously and refer to alkylthiophenes (which also happen to be ‘alkylated thiophenes’) where the total number of carbon atoms of bonded-alkyl group(s) bounded to a member of thiophene ring is either (i) exactly N; or (ii) exactly M or (iii) greater than N and less than M. Analogous definitions (i.e. analogous to ‘alkylthiophenes’) apply to the multi-ring sulfur heterocyclic compounds (i.e. alkylbenzothiophenes and alkyldibenzothiophenes) to the single-ring nitrogen heterocyclic compounds (i.e. alkylpyridines and alkylpyrroles) and to the multi-ring nitrogen heterocyclic compounds (i.e. alkylquinolines, alkylisoquinolines, alkylacridines, and alkylindoles and alkylcarbazoles).

When determining concentration of alkylthiophenes (or, by analogy, alkylbenzothiophenes or alkyldibenzothiophenes or alkylpyridines and alkylpyrroles or alkylquinolines, or alkylisoquinolines or alkylacridines or alkylindoles or alkylcarbazoles), the location to which alkyl group(s) are attached is immaterial.

For the present disclosure, an ‘alkylthiophene-rich mixture’ is a mixture where a majority (or a substantial majority) of the sulfur compounds of the mixture are alkylthiophenes and/or a mixture that is at least 10% or at least 20% by volume alkylthiophene. In embodiments, the ‘alkylthiophene-rich mixture’ is at least 15% wt/wt or at least 20% wt/wt or at least 25% wt/wt sulfur or at least 30% wt/wt sulfur.

For the present disclosure, unless otherwise noted, a ‘boiling point’ refers to an atmospheric boiling point.

For the present disclosure, a ‘highly concentrated mixture’ of CL alkylthiophenes wherein L is a positive integer means that at least 75% wt/wt or at least 90% wt/wt or at least 95% wt/wt or at least 99% wt/wt of the mixture are CL alkylthiophenes.

For the present disclosure, a ‘highly concentrated mixture’ of CL alkylthiophenes wherein K and L are positive integers means that at least 75% wt/wt or at least 90% wt/wt or at least 95% wt/wt or at least 99% wt/wt of the mixture are CK-CL alkylthiophenes.

For the present disclosure, a ‘sulfur-rich feedstock’ or a ‘sulfur-rich pyrolysis liquid’ is at least 3% wt/wt or at least 4% wt/wt sulfur.

For the present disclosure, sulfur-rich type IIs kerogen is at least 6% wt/wt or at least 7% wt/wt or at least 8% wt/wt sulfur.

For the present disclosure, a C_{N}H_{M} hydrocarbons compound or “C_{N}H_{M} hydrocarbons’ refer to compounds having the molecular formula C_{N}H_{M} wherein N and M are both positive integers—N and M may be equal to each other or unequal to each other.

For a mixture comprising multiple compounds, an ‘individual-compound boiling point’ of a given one of the compounds refers to the boiling point of the given compound in its pure form.

For the present disclosure, ‘low temperature pyrolysis’ is pyrolysis that occurs at temperatures of at most 290 degrees Celsius over a period of at least 3 months or at least 6 months or at least 1 year. In some embodiments, ‘low temperature pyrolysis’ occurs between 270 degrees Celsius and 290 degrees Celsius over this period of at least 3 months or at least 6 months or at least 1 year. In some embodiments, ‘low temperature pyrolysis’ occurs between 280 degrees Celsius and 290 degrees Celsius over this period of at least 3 months or at least 6 months or at least 1 year. In this temperature range, pyrolysis of type IIs kerogen proceeds quickly enough to be feasible, while favoring formation of easier-to-hydrotreat species.

For the present disclosure, ‘low severity’ hydrotreating conditions are characterized by (i) a maximum temperature of at most 350 degrees Celsius or at most 340 degrees Celsius or at most 330 degrees Celsius; and (ii) a maximum pressure of at most 120 atmospheres (atm) or at most 110 atm or at most 100 atm or at most 90 atm or at most 80 atm or at most 70 atm.

For the present disclosure, unless otherwise specified, when a feature related to a portion or a fraction of a composition (e.g. of an oil) is disclosed, this refers to by weight (e.g. wt/wt %) and not by mole or by volume. For the present disclosure, unless otherwise specified concentrations and ratios therebetween are by weight (e.g. wt/wt %) and not by mole or by volume.

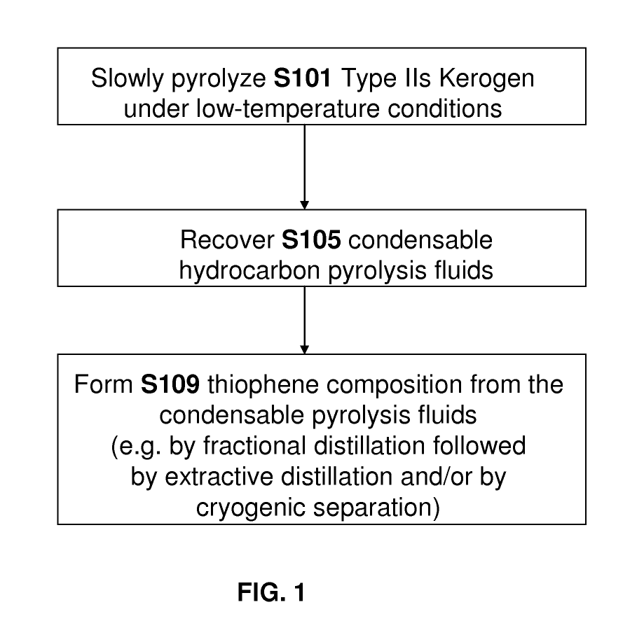

FIG. 1 is a flow chart of a method for manufacturing a thiophenic composition comprising at least 50% wt/wt C1-C3 alkylthiophenes by processing hydrocarbon pyrolysis fluids derived from low-temperature pyrolysis of type IIs kerogen. In non-limiting embodiments, the thiophenic composition may be used as an enhanced oil recovery (EOR) fluid.

In step S101, type IIs kerogen is pyrolyzed under low-temperature conditions. Even though the rate of pyrolysis is slower under these conditions than would be observed at higher temperatures, the resulting hydrocarbon pyrolysis liquids are richer in alkylthiophenes which are useful as an EOR fluid. As will be discussed below, it is believed that the resulting pyrolysis liquids are (i) richer in C1-C3 alkylthiophenes and/or (ii) richer in methyl, di-methyl and/or tri-methyl thiophenes.

In step S105, the pyrolysis fluids comprising condensable hydrocarbon fluids are recovered—e.g. via production wells for the case of in situ pyrolysis of sulfur-rich type IIs kerogen. In step S109, a thiophenic composition comprising at least 50% wt/wt C1-C3 alkylthiophenes is formed from the condensable hydrocarbon pyrolysis fluids.

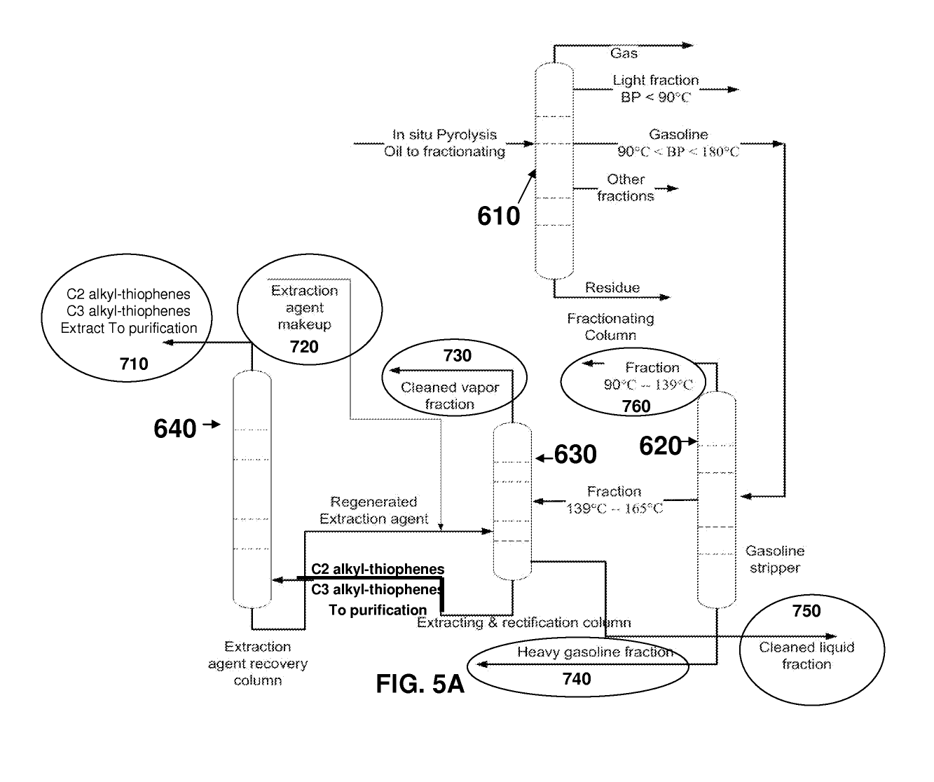

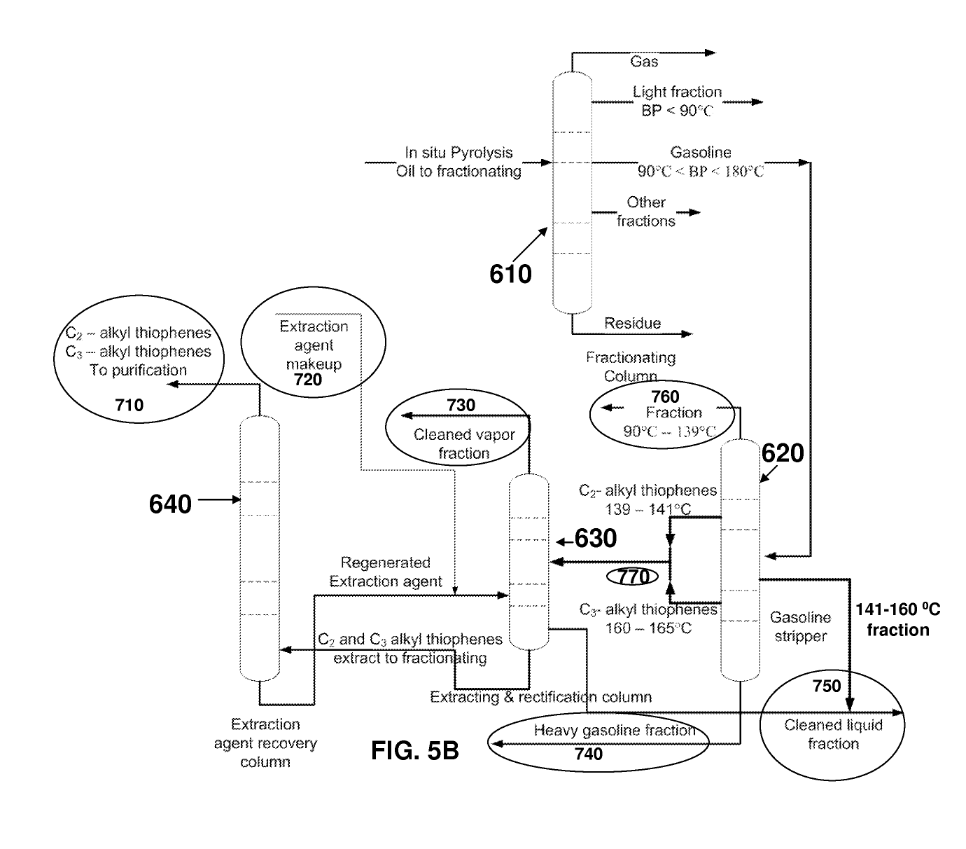

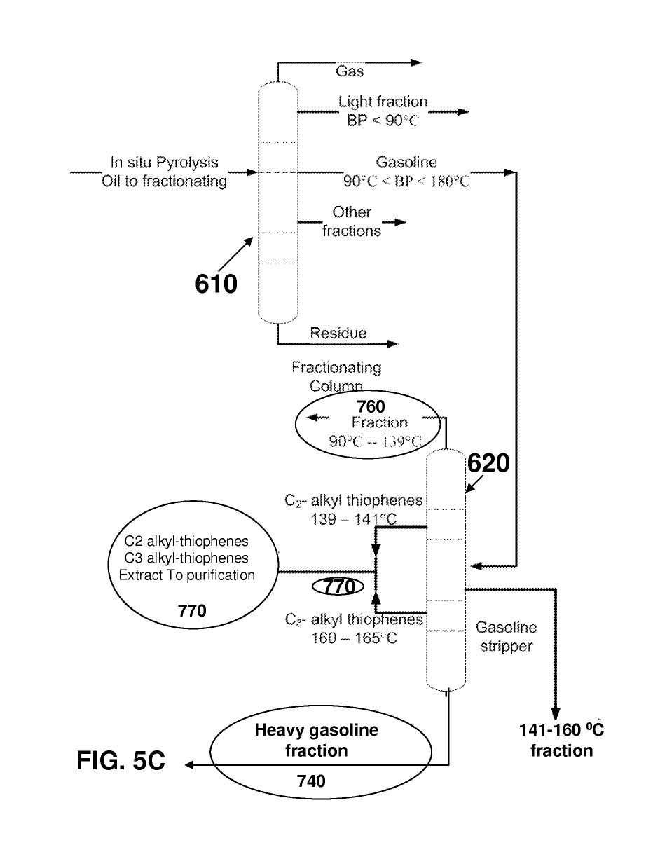

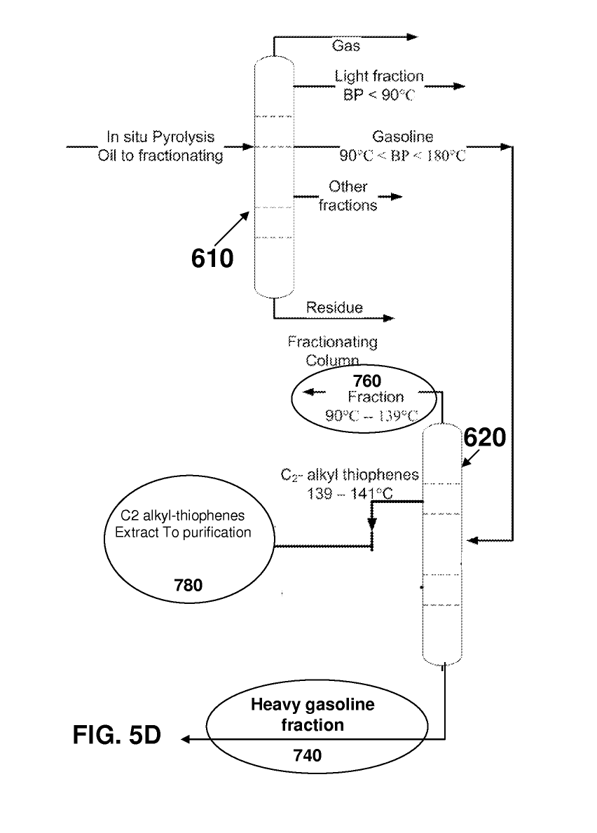

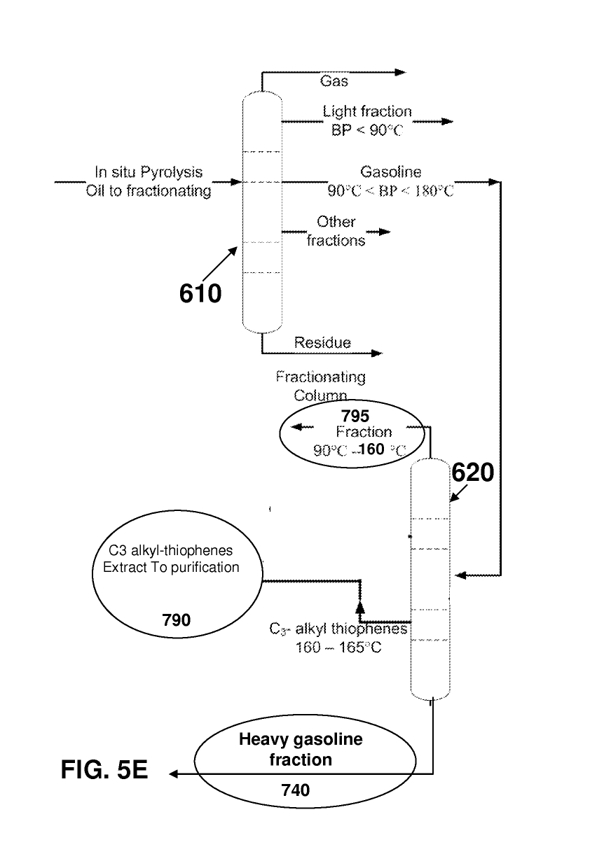

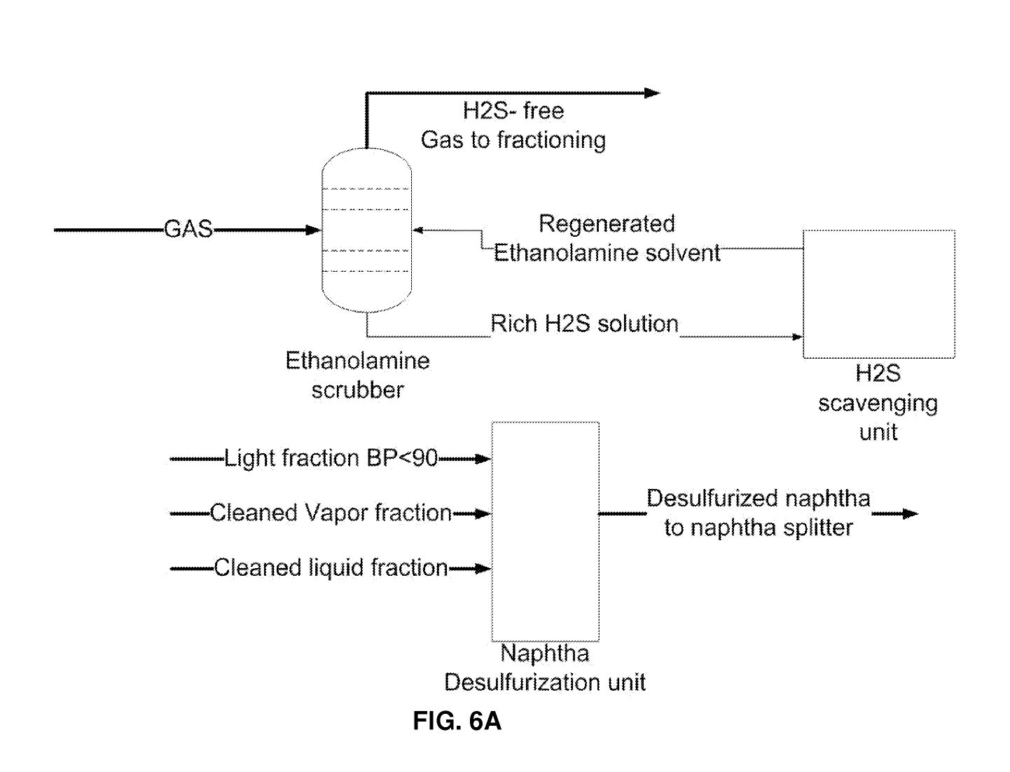

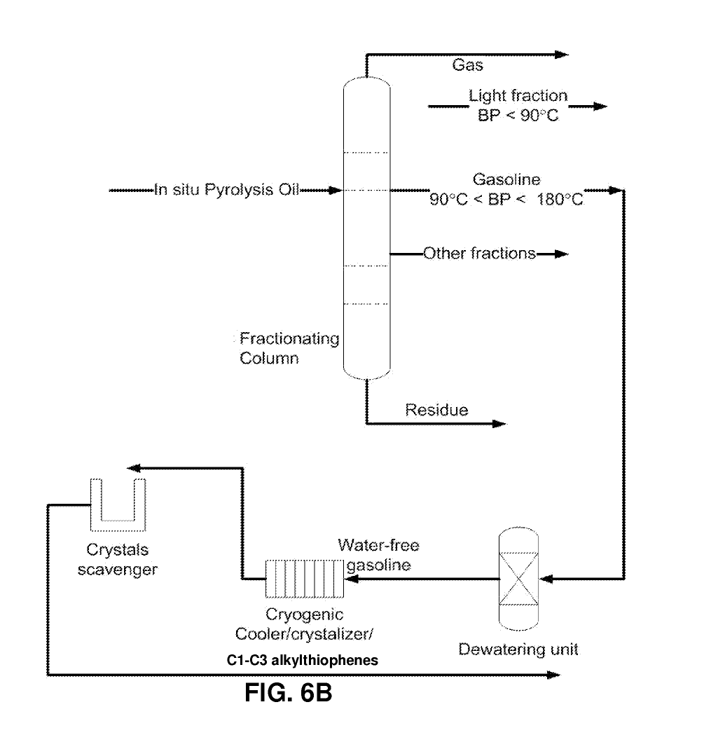

In non-limiting embodiments, step S109 may include at least one of (i) a fractional distillation; (ii) an extractive distillation; and a (iii) a cryogenic separation. For example, as illustrated below in FIGS. 5-6, an extractive distillation and/or a cryogenic separation may follow the fractional distillation.

For example, the primary purpose of step S109 may be to separate C1-C3 alkylthiophenes (or any component thereof) from C_{N}H_{M} hydrocarbons (N and M are both positive integers). The C1-C3 alkylthiophenes (or any component thereof) may be used as an EOR fluid (or for any other purpose)—in addition, step S109 may reduce the cost of hydrotreating the pyrolysis-derived oil comprising the C_{N}H_{M} hydrocarbons.

Examples of apparatus for performing step S109 are disclosed below with reference to FIGS. 5-6.

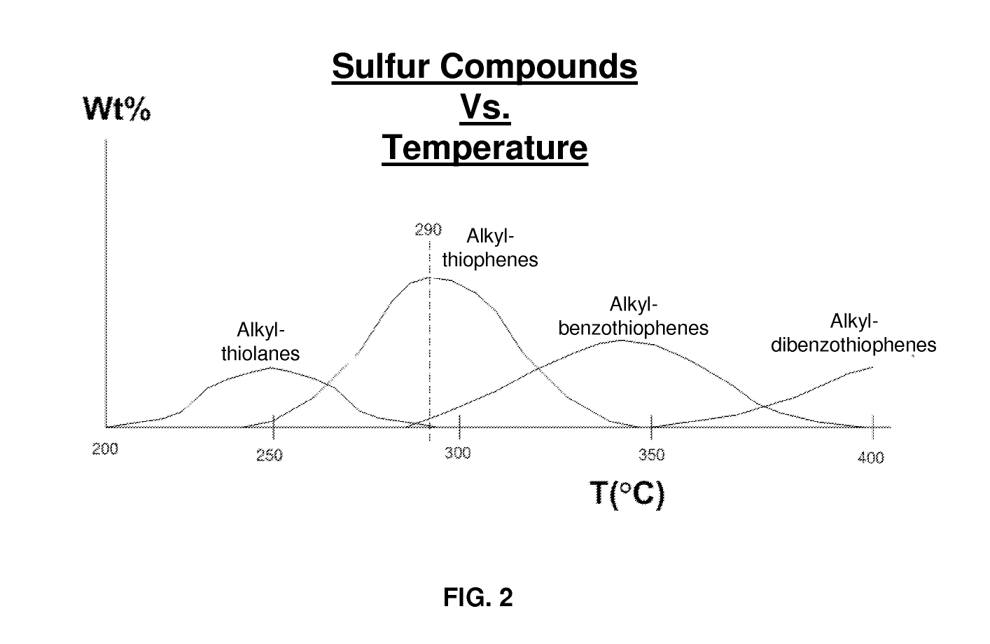

FIG. 2 illustrates the wt % of sulfur compounds within pyrolysis formation liquids derived from type IIs kerogen as a function of temperature according to one example. Sulfur compounds within formation fluids generated at very low pyrolysis temperatures (220-270 degrees Celsius) are primarily alkylthiolanes. Sulfur compounds within formation fluids generated at low pyrolysis temperatures (260-320 degrees Celsius) are primarily alkylthiophenes. Sulfur compounds within formation fluids generated at higher and more conventional pyrolysis temperatures are primarily alkylbenzothiophenes (320-370 degrees Celsius) or alkyldibenzothiophenes (370-400 degrees Celsius).

As shown in FIG. 2, a majority, or significant majority, or substantially all sulfur species in pyrolysis fluids formed between 270 and 290 degrees Celsius are thiophene or alkylthiophenes.

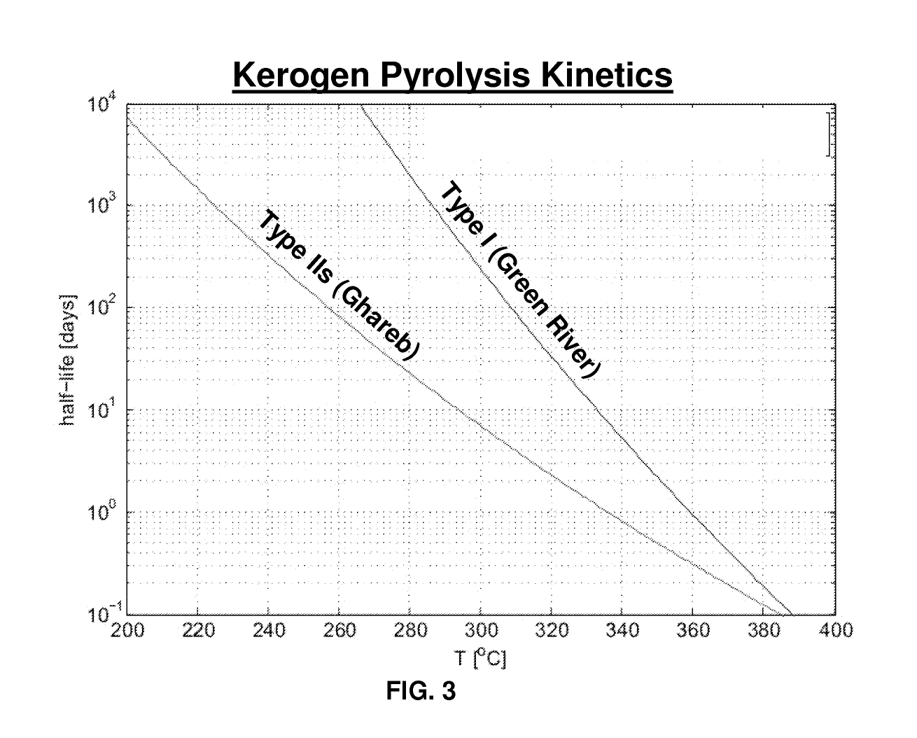

As discussed below in examples below, the present inventors have conducted kinetics experiments related to kerogen pyrolysis kinetics. Results are presented in FIG. 3. In particular, in FIG. 3 the pyrolysis kinetics of type IIs kerogen is compared to that of type I Green River kerogen. From FIG. 3, one may conclude that at 290 degrees Celsius, the pyrolysis of type IIs kerogen is surprisingly about two orders of magnitude faster than pyrolysis of type I Green River kerogen. Thus, pyrolysis at this low temperature may be surprisingly viable.

Many sulfur-rich hydrocarbons are sourced from a subset of Type II kerogen known to be sulfur-rich, called Type IIs or IIs. A schematic representation of one type of organic matter in Type IIs kerogen is illustrated below:

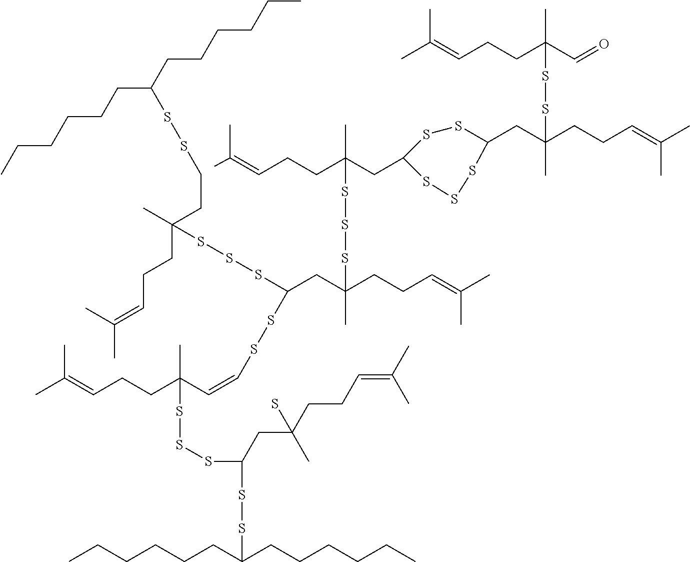 [SMILES: CCCCCCC(CCCCCC)SSCCC(C)(CCC=C(C)C)SSSC(CC(C)(CCC=C(C)C)SSSC(C)(CCC=C(C)C)CC1SSSC(CC(C)(CCC=C(C)C)SSC(C)(C=O)CCC=C(C)C)SS1)SS/C=C\C(C)(CCC=C(C)C)SSSC(CC(C)(S)CCC=C(C)C)SSC(CCCCCC)CCCCCC]

During lower-temperature pyrolysis, the pyrolysis liquids have both a higher alkylthiophene as well as a sulfur content, since most bonds are broken at lower temperatures tend to be S—S bonds. In this sense, pyrolyzing type IIs at lower temperatures may be more advantageous.

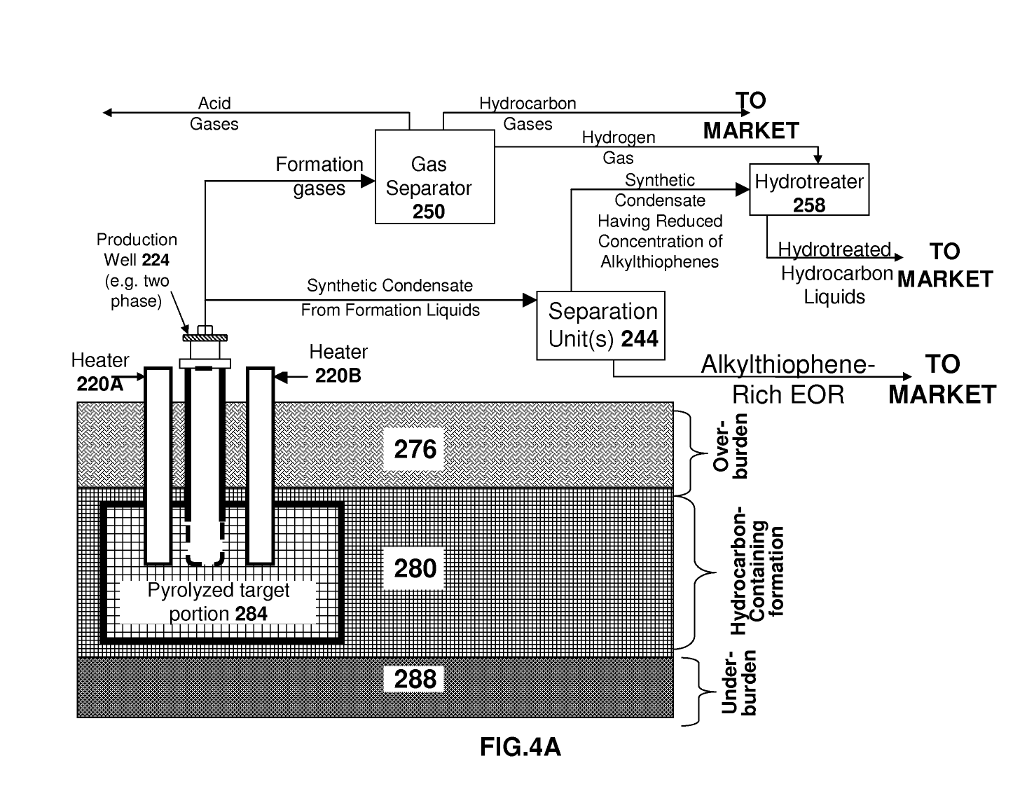

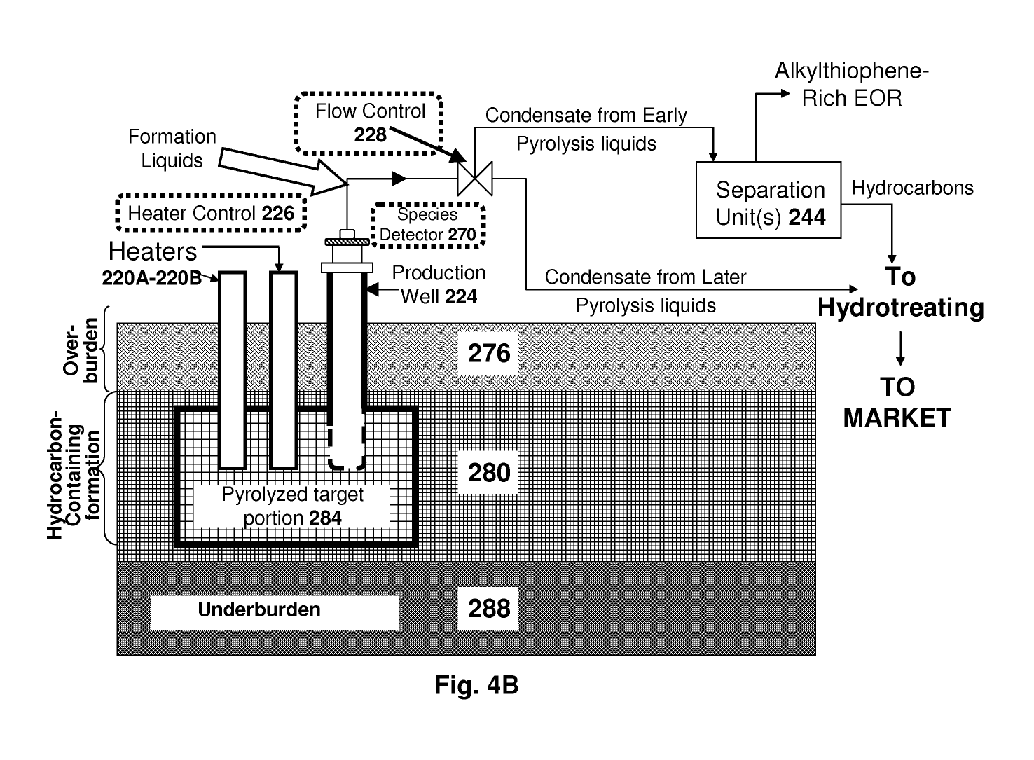

FIG. 4A illustrates a system for manufacturing an alkylthiophene-based EOR fluid by in situ pyrolysis of type IIs kerogen. Subsurface heaters 220 are operated to pyrolyze a target portion 284 of a subsurface hydrocarbon-bearing formation—e.g. a kerogenous-chalk containing type IIs kerogen. Formation gases are recovered via production well(s) 224 and subjected to a gas separation in gas separator 250. A synthetic condensate from pyrolysis formation liquids is processed in a separation unit(s) 244—for example, fractional distillation followed by extractive distillation. This yields (i) an alkylthiophene-based EOR fluid and (ii) a hydrocarbon synthetic condensate having a reduced concentration of alkylthiophenes.

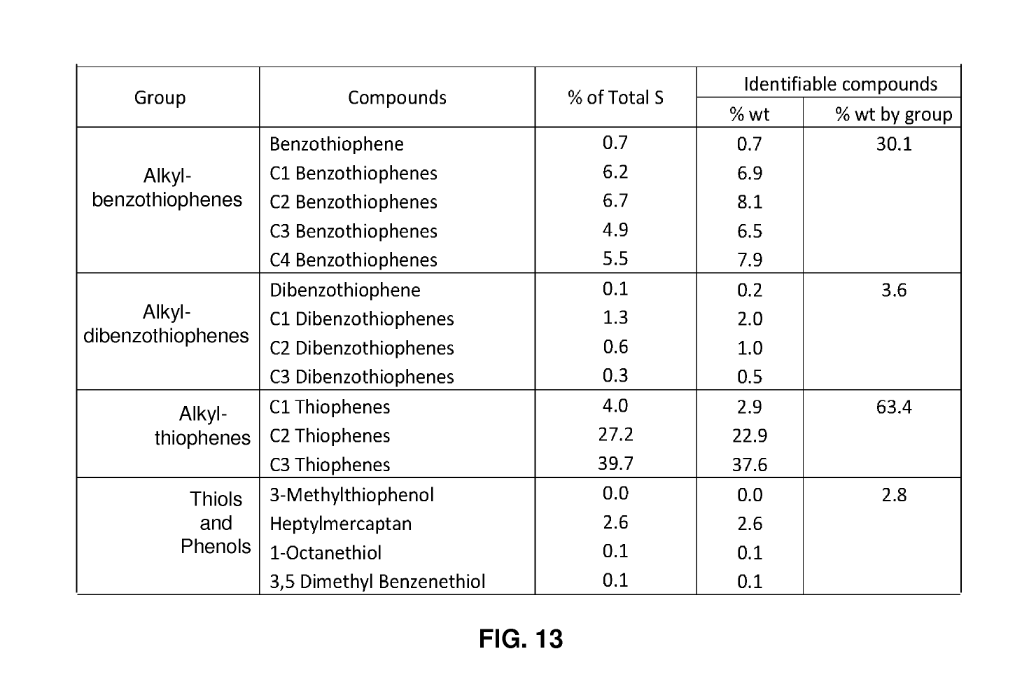

When pyrolysis occurs at relatively low temperatures, and as discussed below with reference to FIG. 13, a majority of the sulfur compounds of the hydrocarbon pyrolysis liquids are, in fact, alkylthiophenes. Not only is it possible to economically recover relatively large quantities of alkylthiophenes, but doing so may reduce the amount of hydrotreatment required to convert hydrocarbon pyrolysis liquids into low-sulfur oil or derivatives (e.g. transportation fuel) thereof.

As noted above with reference to FIG. 2, at low pyrolysis temperatures the concentration of multi-ring sulfur heterocycles, within hydrocarbon pyrolysis liquids, is relatively low. In one example, it is possible to only pyrolyze at these low temperatures. This may reduce the need for fractional distillation. Alternatively, as illustrated in FIG. 4B, it is possible to perform some pyrolysis at lower temperatures and some pyrolysis at higher temperatures. In the example of FIG. 4B, a flow control 228 separates condensate from early pyrolysis liquids (i.e. formed in ‘earlier’ stages of pyrolysis at lower temperatures) from condensate from later pyrolysis liquids (i.e. formed in ‘later’ staged of pyrolysis at lower temperatures). The former is rich in alkylthiophenes and may be fed to separation unit(s) 244 to manufacture an alkylthiophene-based EOR fluid.

The flow control apparatus 228 may be operated by detecting species concentrations within the pyrolysis liquids in any manner (e.g. by spectrometry or by chromatography).

Although FIGS. 4A-4B relate to the specific case of in situ pyrolysis, this is not limiting. Alternatively, pyrolysis may be carried out in a pit or impoundment or any enclosure (e.g. excavated enclosure) under anoxic conditions. For example, the pyrolysis within the enclosure may be carried out slowly and at relative low-temperature conditions.

FIGS. 5A-5E illustrate apparatus for forming, from pyrolysis fluids, a thiophene composition comprising at least 50% wt/wt or at least 70% wt/wt or at least 95% wt/wt or at least 99% wt/wt CK-CL alkylthiophenes, wherein K and L are both positive integers equal to at most 3, L>K.

Pyrolysis oil/hydrocarbon pyrolysis fluids are fed into fractionating column 610 to obtain a thiophene-hydrocarbon mixture comprising: (i) CK-CL alkylthiophenes, wherein K and L are both positive integers equal to at most 3, L>K; and (ii) C_{N}H_{M} hydrocarbons compounds (N and M are both positive integers) having a similar boiling points.

The thiophene-hydrocarbon mixture may be further processed (e.g. in extracting and rectification column 620 and extraction agent recovery column 630) to obtain: (i) hydrocarbon fractions 730, 740, 750, 760 comprising primarily C_{N}H_{M} hydrocarbons compounds; and (ii) a thiophenic fraction 710 comprising primarily thiophenic compounds. The hydrocarbon fractions may be hydrotreated while the thiophenic fraction may be used as an EOR fluid or for any other application.

One extraction agent that may be used in column 630 is N-Methyl-2-pyrrolidone (NMP). NMP has a boiling point of about 203 degrees Celsius, and belongs to the class of dipolar aprotic solvents which includes also dimethylformamide, dimethylacetamide and dimethyl sulfoxide. Other names for this compound are: 1-methyl-2-pyrrolidone, N-methylpyrrolidone, N-methylpyrrolidinone and the brand name Pharmasolve.

The apparatus of FIG. 5A is arranged so that a concentration of CK-CL alkylthiophenes in the thiophenic fraction 710 is significantly larger than within the input pyrolysis oil. The apparatus of FIG. 5A is arranged so that a concentration of CK-CL in the hydrocarbon fractions 730, 740, 750, 760 is significantly less than in the input pyrolysis oil.

The non-limiting example of FIG. 5A is arranged to produce a mixture 710 of C2 and C3 alkylthiophenes. In the example of FIG. 5A, there is a need in column 630 to separate the C2 and C3 alkylthiophenes from C_{N}H_{M} hydrocarbons compounds having boiling points over the 139-165 degrees Celsius range. For this boiling point range, a difference between a maximum and a minimum thereof is over 25 degrees Celsius, and a variety of C_{N}H_{M} hydrocarbons compounds may be fed to column 630.

Alternatively as shown in FIG. 5D, if the goal is to obtain a highly concentrated mixture of di-methyl alkylthiophenes without significant quantities of C3 alkylthiophenes, it is possible to operate column 630 to extract only compounds having a boiling point of around 140 degrees Celsius—e.g. in the much more narrow range between 139 degrees Celsius and 141 degrees Celsius. One advantage of working in this manner is that it is possible, by fractional distillation, to produce having a higher or significantly higher concentration of alkylthiophenes since fewer C_{N}H_{M} hydrocarbons compounds having boiling points in the more narrow range. Another advantage of working in this manner is that it may be possible to produce a highly-concentrated thiophenic mixture by relying only on fractional distillation or on cryogenic separation of thiophenic compounds from other hydrocarbons.

Similarly, as shown in FIG. 5E. if the goal is to obtain a highly concentrated mixture of tri-methyl alkylthiophenes without significant quantities of C2 alkylthiophenes, it is possible to work in the range between about 160 degrees Celsius and 165 degrees Celsius.

In some embodiments, as shown in FIG. 5B-5C it is possible to separately distill the C2 and C3 alkylthiophenes to a relatively narrow boiling point range and then to subsequently mix together the C2 and C3 alkylthiophene compositions to form a highly concentrated mixture of C2-C3 alkylthiophenes. This may be advantageous to the arrangement illustrated in FIG. 5A, and may obviate the need (see FIG. 5C) for a subsequent separation/distillation step after the fractional distillation.

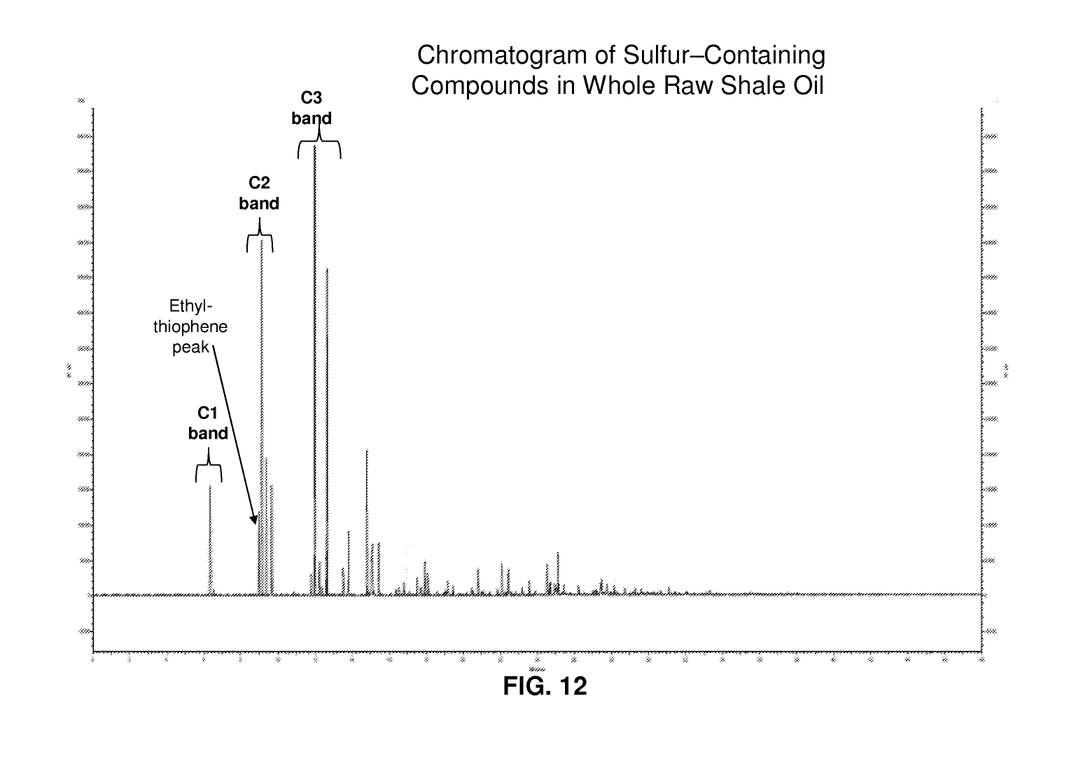

The skilled artisan will appreciate that FIGS. 12-13 shows that the pyrolysis liquids derived low temperature-pyrolysis of type IIs kerogen are surprisingly rich in C1, C2 and C3 alkylthiophenes. The present inventors are now illustrating methods for providing a thiophenic solution having even greater concentrations of C1, C2 and/or C3 alkylthiophenes.

Experiments commissioned by the present inventors have indicated that most alkylthiophenes in pyrolysis-liquids formed at low temperatures tend to be lower-molecular-weight alkylthiophenes—e.g. methyl, di-methyl, and tri-methyl thiophenes.

The table below describes some properties of these species.

Table of Densities and Boiling Points for Methylthiophenes

| | | Density | Boiling Point |
|---|---|---|---|
| Chemical | Formula | (g/cc) | (° C.) |
| Thiophene | C4H4S | 1.051 | 84 |
| 2-methylthiophene | C5H6S | 1.014 | 113 |
| 3-methylthiophene | C5H6S | 1.016 | 115-117 |
| 2,3 dimethylthiophene | C6H8S | 1.002 | 140-141 |
| 2,4 dimethylthiophene | C6H8S | 0.994 | 139-141 |
| 2,5 dimethylthiophene | C6H8S | 0.985 | 139-141 |
| 2,3,5 trimethylthiophene | C7H10S | 0.980 | 161-163 |

Embodiments of the present invention relate to a thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) at least a majority or at least a substantial majority or substantially all of the alkylthiophenes of the composition are derived from pyrolysis of type IIs kerogen.

There are a number of markers/fingerprints that are indicative that alkylthiophenes of the thiophenic composition are derived from pyrolysis of type IIs kerogen.

For example, a δ³⁴S(‰) value of the composition may be at least +0.75 or at least +1.0 or at least +1.25 or at least +1.5, the δ³⁴S(‰) value describing deviations from the V-CFT (Vienna Canyon Diablo Troilite) standard—see Geochimica et Cosmochimica Acta, Vol. 69, No. 22, pp. 5317-5331, 2005

Another possible marker is a presence of olefins from pyrolysis. In another example, an inorganic element may be used—for example, the thiophenic composition may include at least 10 PPM or at least 20 PPM silicon.

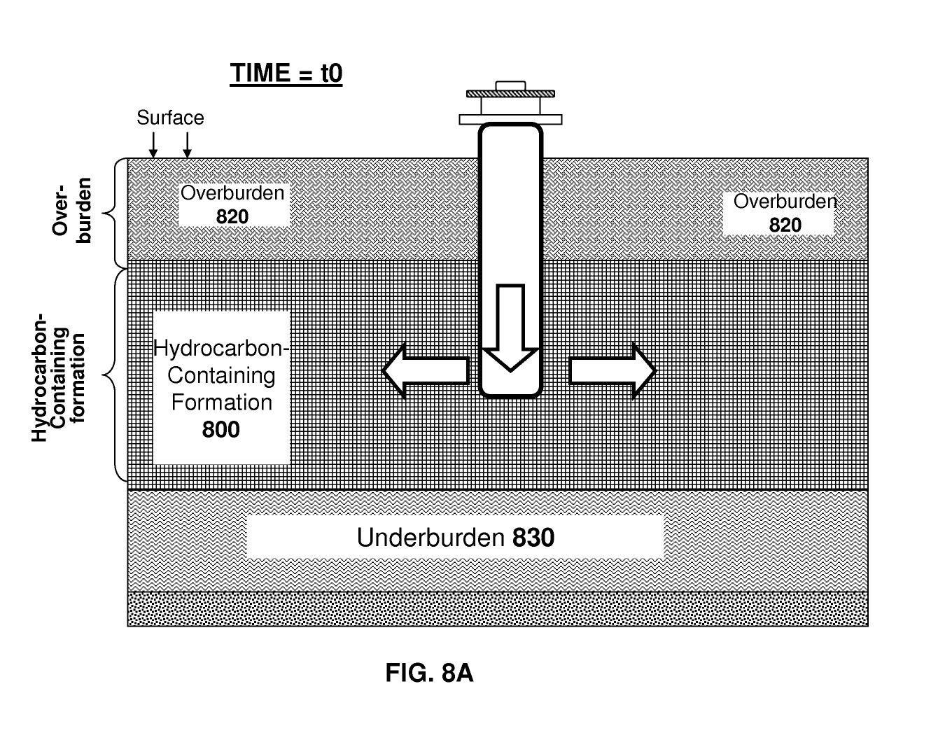

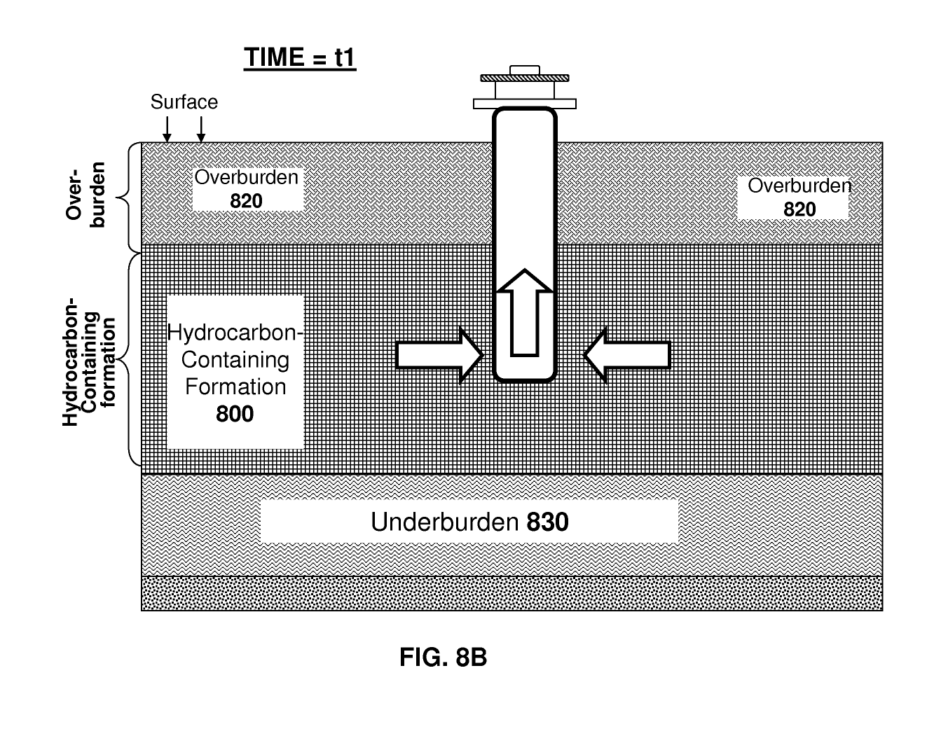

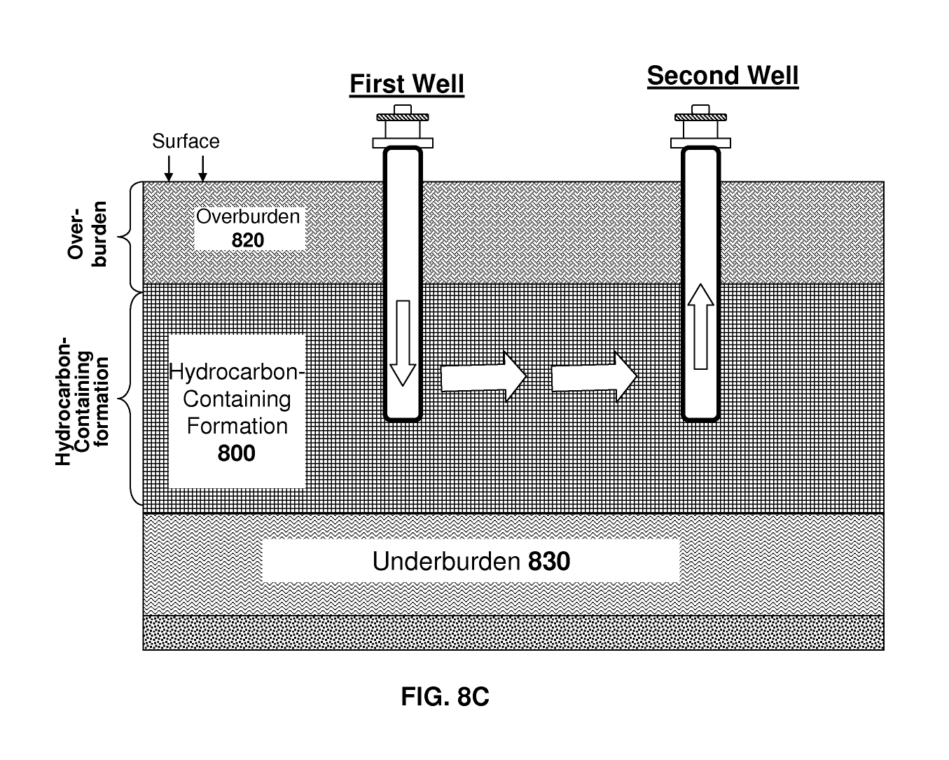

FIGS. 7-8 relate to the utilization of the alkylthiophene-based EOR fluid. In step S201 of FIG. 7, the EOR fluid is injected into a hydrocarbon-containing subsurface formation—e.g. a tar sands formation. In step S205, a mixture comprising oil or bitumen together with at least some of the injected alkylthiophenes is recovered—e.g. via production wells. This mixture may have a relatively low naphtha content. As such, it is possible that fractional distillation is sufficient in step S209 when separating out alkylthiophenes from the recovered bitumen or oil. The separating step of S209 has two advantages: (i) it obviates the need to hydrotreat the alkylthiophenes mixed in with oil or bitumen and (ii) it allows for re-use of these alkylthiophenes as an EOR agent.

In step S213, the recovered alkylthiophenes (e.g. recovered from the produced oil or bitumen by fractional distillation) are re-injected into the formation. (e.g. tar sands formation).

FIGS. 8A-8B relates to a ‘huff-and-puff’ usage of the recovery fluids. FIG. 8C relates to flow of the recovery fluid between multiple wells.

The method may be practiced as a ‘huff-n-puff’ or cyclical injection and production method—for example, see FIGS. 8A-8B. There may be one cycle of injection and production, two cycles, or N cycles, where N may be at least 5, or at least 10, or at least 15, or at least 20, or more cycles of injection and production. The EOR fluid may be heated during injection.

### EXAMPLES

The above description is not intended to limit the claimed invention in any manner; furthermore, the discussed combination of features might not be absolutely necessary for the inventive solution.

The present invention will be further illustrated in the following examples. However it is to be understood that these examples are for illustrative purposes only, and should not be used to limit the scope of the present invention in any manner

### Example 1

### Type IIs Kerogen

An 8.6 cm diameter (3.4 inch) PQ core sample of type IIs kerogen was cored from a kerogenous chalk with the following petrophysical properties: porosity of 35-40%, permeability of 0.05-0.2 mD, and total organic carbon (TOC) of 14-18 wt %.

A Fischer Assay was performed in which 100 grams of the raw rock were crushed to <2.38 mm pieces, heated in a vessel to 500° C. at a rate of 120° C./min, and held at that temperature for 40 minutes. The distilled vapors of oil, gas, and water were condensed and centrifuged to assess the amount of oil yielded by the rock sample. Fischer Assay results for the oil shale is 24-29 gal/ton.

Elemental analysis of the kerogenous chalk sample from the Ghareb formation, a bituminous and kerogenous chalk, gave the kerogen composition presented in the table below.

Kerogen composition in wt %

| | |
|---|---|
| Carbon | 65.30 |
| Hydrogen | 7.95 |
| Nitrogen | 2.15 |
| Oxygen | 14.36 |
| Sulfur | 9.80 |

The high sulfur content indicates that this is a type IIs kerogen.

### Example 2

### Slow Pyrolysis of Samples of Type IIs Kerogenous Chalk Simulating In Situ Pyrolysis

First, Fischer Assay numbers were collected from the samples, then the API gravity of the Fischer Assay oil was measured. All measurements were reported on a dry weight basis. Samples of type IIs kerogen-bearing oil shale was crushed to 1-5 mm pieces and packed into a retort. The retort vessel chosen was a pressure-regulated semi-batch pyrolysis reactor.

The weight change of the retort system was tared, then measured every 1.5 hours. Flow measurements were also made. A gas chromatograph (GC) was run every 1.5 hours, timed to be coincident with the weight and flow measurements, to identify compounds in the pyrolysis fluids. The H₂S level was measured with a Draeger tube, a colorimetric gas detection technology, downstream of the reactor and GC.

Approximately 30 experimental runs were conducted. The temperature ramps and the constant pressure for the system during a single run were varied from one run to another according to the inventors' specifications. Temperature ramps ranged from 1-4° C./hr starting from ambient temperature increasing to no higher than 430° C. with a back pressure on the system held constant at a pressure chosen from between 0-150 psig.

For example, an experiment held at 150 psig for the duration of the experiment and with a maximum temperature of 430° C., with a 1-2° C. rate was conducted as follows. The reactor/retort was heated at a rate of 1° C./min on the skin temperature up to 175° C. and held at that temperature for 1 hour minimum. From 175° C., the temperature was increased by 2° C./hr on the skin temperature until the skin temperature reached 200 ° C. The retort was held at this temperature until the center shale temperature reached 200° C. (Free water boils at 185° C., so the reactor pressure was carefully adjusted and from this point on, the top head heater of the retort was held at a 5-10° C. hotter temperature in order to prevent water vapors from condensing on the head.) A water product receiver was weighed every 3 hours until all of the water was removed from the retort system. Beyond 200° C., heating continued at 2° C./hr on the skin temperature. Gas was collected on another product receiver, which was also tared and weighed. When the system reached 300° C., the weight and volume of oil and water removed are measured. Oil and water were held in reserve in a sealed refrigerated container. Product collection continued with a separate product receiver. When the mid-retort shale temperature reached 430° C., the temperature was held for at least 8 hours with only the head temperature held 10° C. higher. When the gas flow was reduced to a negligible level, all retort heaters were turned off. As soon as the pressure measured decreased, purging with N₂ or Argon, allowed for obtaining the final oil product collection. The retort was checked for residual oil. Product samples were stored in sealed refrigerated containers. The spent shale was weighed and used to perform three Fischer Assays to compare with the initial Fischer Assay.

This procedure was performed in the same manner for samples at other pressures and temperature ramps. The samples collected from this experiment were also subjected to elemental analysis and will be discussed further below.

### Example 3

### Sulfur Specification in Pyrolysis Liquids

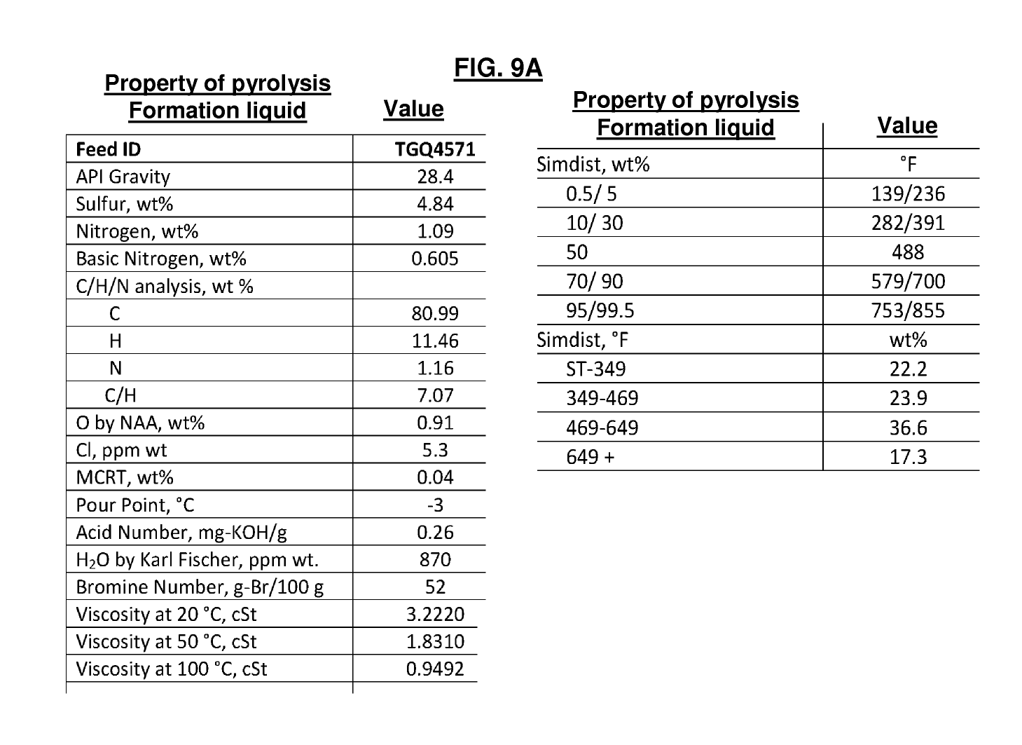

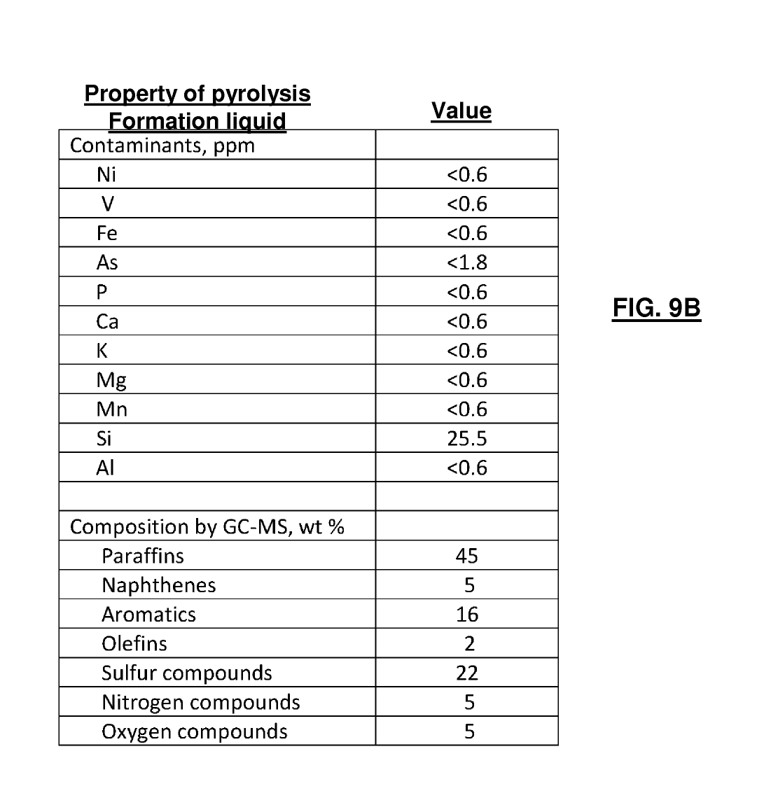

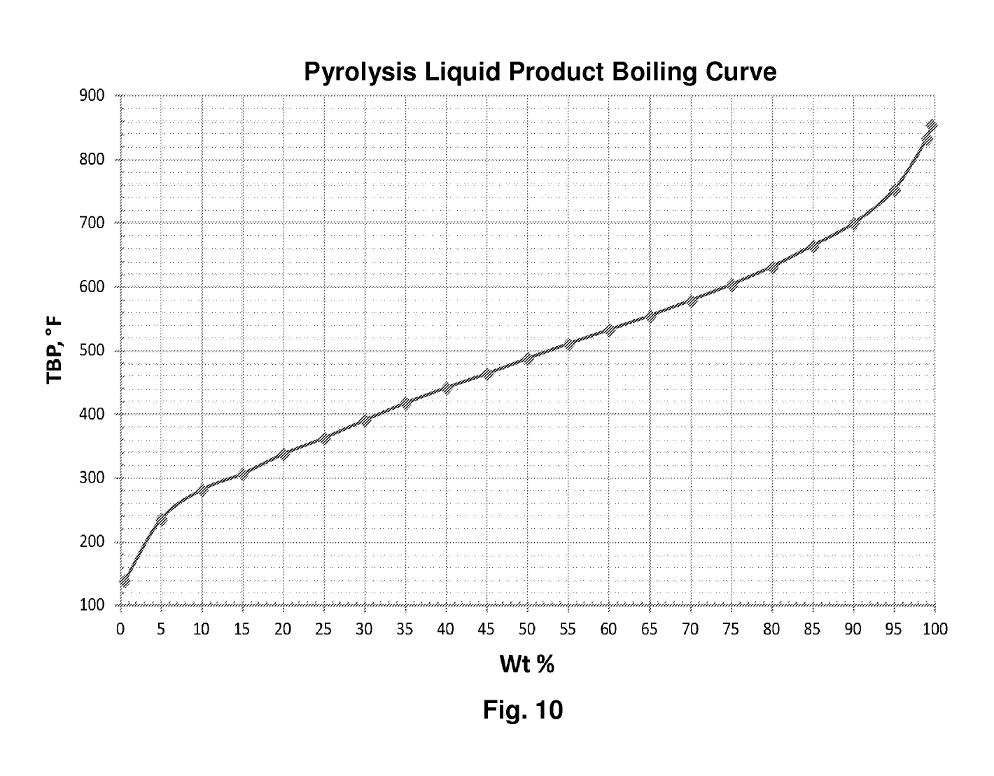

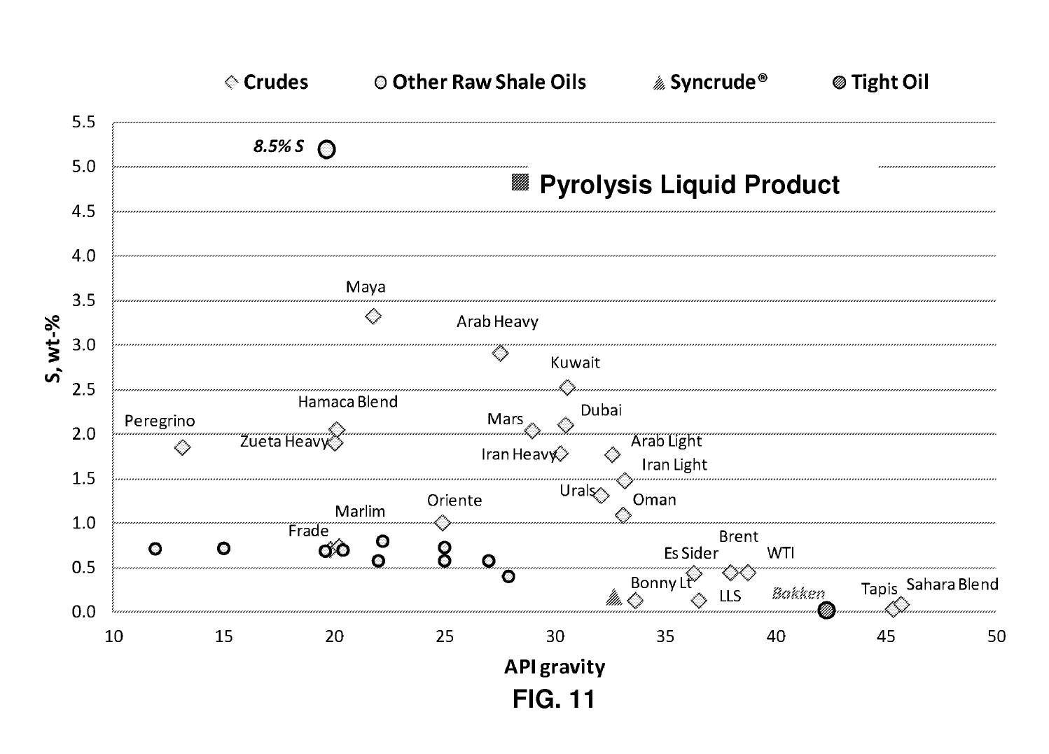

The pyrolysis liquid products from the various temperatures and pressures were blended to create a more accurate representation of product in the field. The properties of pyrolysis liquids blended from the aliquots collected in the procedure described above are given in FIGS. 9A and 9B. A boiling curve derived from simulated distillation data is shown in FIG. 10. The material was relatively light and liquid at room temperature. In spite of its relatively low end point, it contains very high concentrations of sulfur and nitrogen (4.84 and 1.09 wt %, respectively). This is contrary to what is frequently seen in petroleum feedstocks and in several other shale oils, as clearly shown in FIG. 11. Additional characterization tests were run on the hydrocarbon pyrolysis liquid product, attempting to accurately identify the main types of compounds present. GCxGC data (not shown here) qualitatively showed that saturates are most abundant. Sulfur-containing compounds, such as thiophenes, were also very significant. The feed was also characterized by GC-MS to obtain more quantitative composition data. The results are part of FIGS. 9A and 9B. While significant, the concentration of aromatic hydrocarbon compounds was relatively low compared with values commonly observed in other shale oils and may be related to the process used to generate the hydrocarbon pyrolysis liquid product.

Chromatography tests using a Pulse Flame Photometric Detector (PFPD) optimized for sulfur detection were performed to determine the identity of the sulfur-containing compounds in the hydrocarbon pyrolysis oil product (see FIG. 12). The concentrations of identifiable compounds derived from GC peak areas are summarized in FIG. 13. The majority of the sulfur compounds are thiophenes (including alkyl thiophenes). Benzothiophenes are the second most significant group. Most of these compounds are relatively light, with molecules containing between 5 and 12 carbon atoms. 63.4% were alkylthiophenes with 22.9% C2 alkylthiophenes and 37.6% C3 alkylthiophenes.

Additional S-speciation analysis showed that the C1, C2 and C3 alkylthiophenes were predominantly methyl, dimethyl and trimethyl alkylthiophenes.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) at least a majority or at least a substantial majority or substantially all of the alkylthiophenes of the composition are derived from pyrolysis of type IIs kerogen.

2. A thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) a δ³⁴S(‰) value of the composition is at least +0.75 or at least +1.0 or at least +1.25 or at least +1.5, the δ³⁴S(‰) value describing deviations from the V-CFT (Vienna Canyon Diablo Troilite) standard.

3. The composition of any preceding claim, wherein K=2 and L=3.

4. A thiophenic composition comprising at least 50% wt/wt or at least 70% wt/wt or at least 95% wt/wt or at least 99% wt/wt CL alkylthiophenes, wherein L is a positive integer equal to at most 3, and at least a majority or at least a substantial majority or substantially all of the alkylthiophenes of the mixture are derived from pyrolysis of type IIs kerogen.

5. A thiophenic composition comprising at least 50% wt/wt or at least 70% wt/wt or at least 95% wt/wt or at least 99% wt/wt CL alkylthiophenes, wherein L is a positive integer equal to at most 3, and a δ³⁴S(‰) value of the composition is at least +0.75 or at least +1.0 or at least +1.25 or at least +1.5, the δ³⁴S(‰) value describing deviations from the V-CFT (Vienna Canyon Diablo Troilite) standard.

6. The composition of any of claims 4-5 wherein a value of L is 1.

7. The composition of any of claims 4-5 wherein a value of L is 2.

8. The composition of any of claims 4-5 wherein a value of L is 3.

9. The composition of any previous claim, comprising at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

10. A thiophenic composition comprising at least 50% wt/wt or at least 70% wt/wt or at least 95% wt/wt or at least 99% wt/wt CK-CL alkylthiophenes, wherein (i) K and L are both positive integers equal to at most 3, L>K and (ii) the composition comprises at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

11. The composition of claim 10 wherein K=2 and L=3.

12. A thiophenic composition comprising at least 50% wt/wt or at least 70% wt/wt or at least 95% wt/wt or at least 99% wt/wt CL alkylthiophenes, wherein L is a positive integer equal to at most 3, and the composition comprises at least 0.1% wt/wt or at least 0.3% wt/wt or at least 0.5% wt/wt or at least 1% wt/wt a polar organic solvent having a boiling point of at least 160 degrees Celsius or at least 180 degrees Celsius.

13. The composition of claim 12 wherein a value of L is 1.

14. The composition of claim 12 wherein a value of L is 2.

15. The composition of claim 12 wherein a value of L is 3.

16. The composition of any of claims 9-15 wherein the polar organic solvent is capable of selectively extracting methylthiophenes, dimethylthiophenes and trimethylthiophenes from a liquid mixture involving liquid-phase C_{N}H_{M} hydrocarbon compounds.

17. The composition of any of claims 9-15 wherein the polar organic solvent is capable of selectively extracting C1-C3 alkylthiophenes,from a liquid mixture involving liquid-phase C_{N}H_{M} hydrocarbon compounds.

18. The composition of any of claims 9-17 wherein (i) a boiling point of the organic solvent is at least 180 degrees Celsius or at least 190 degrees Celsius and/or the organic solvent is NMP.

19. The composition of any of claims 9-17 wherein the organic solvent is immiscible with water.

20. The composition of any previous claim, comprising at least 0.1% wt/wt or at least 0.3% wt/wt or at least 0.5% wt/wt or at least 1% wt/wt or at least 3% wt/wt or at least 5% wt/wt or at least 10% wt/wt C_{N}H_{M} hydrocarbon compounds, wherein an individual-compound atmospheric boiling point of each C_{N}H_{M} hydrocarbon compound is between about 80° C. and about 175° C.

21. A thiophenic composition comprising at least 50% wt/wt or at least 70% wt/wt or at least 95% wt/wt or at least 99% wt/wt CL alkylthiophenes, wherein L is a positive integer equal to at most 3, and the composition comprises at least 0.1% wt/wt or at least 0.3% wt/wt or at least 0.5% wt/wt or at least 1% wt/wt or at least 3% wt/wt or at least 5% wt/wt or at least 10% wt/wt C_{N}H_{M} hydrocarbon compounds.

22. The composition of any of claims 20-21 wherein the individual-compound atmospheric boiling point of each C_{N}H_{M} hydrocarbon compound is at least 110° C. or at least 135° C. or at least 155° C.

23. The composition of any of claims 20-21 wherein the individual-compound atmospheric boiling point of each C_{N}H_{M} hydrocarbon compound (i) matches that of methyl-thiophenes, dimethyl-thiophenes and tri-methyl-thiophenes and/or (ii) has a value between about 113° C. and about 117° C. or between 139° C. and about 141° C. or between about 161° C. and about 163° C.

24. The composition of any preceding claim, comprising at least 50% wt/wt or at least 70% wt/wt or at least 95% wt/wt or at least 99% wt/wt methylthiophenes.

25. The composition of any preceding claim, comprising at least 50% wt/wt or at least 70% wt/wt or at least 95% wt/wt or at least 99% wt/wt dimethylthiophenes.

26. The composition of any preceding claim, comprising at least 50% wt/wt or at least 70% wt/wt or at least 95% wt/wt or at least 99% wt/wt trimethylthiophenes.

27. The composition of any preceding claim, comprising at least 99% wt/wt dimethylthiophenes.

28. The composition of any preceding claim derived from pyrolysis of type IIs kerogen of a kerogeneous chalk

29. The composition of any preceding claim derived from pyrolysis of type IIs kerogen of a Ghareb formation kerogeneous chalk.

30. The composition of any preceding claim comprising at least 10 PPM or at least 25 PPM or at least 50 PPM or at least 100 PPM or at least 0.25% wt/wt olefins.

30. The composition of any preceding claim comprising at least 10 PPM silicon.

31. A method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 139 degrees Celsius and 141 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

32. A method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 160 degrees Celsius and 165 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

33. A method of processing an oil, the method comprising:
a. separating, from an oil, a thiophene-rich composition comprising primarily CL alkylthiophenes and further comprising C_{N}H_{M} hydrocarbons where N and M are positive integers and individual-component boiling points are substantially between 115 degrees Celsius and 118 degrees Celsius;
b. processing the thiophene-rich mixture to remove therefrom a majority of alkylthiophenes so as to yield a hydrocarbon-rich mixture comprising (i) at most 5% wt/wt or at most 3% wt/wt or at most 1% wt/wt alkylthiophenes and (ii) comprising primarily the boiling-point C_{N}H_{M} hydrocarbons;
hydrotreating the hydrocarbon-rich mixture or a derivative thereof.

34. A method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 115 degrees Celsius and at most 175 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C1-C3 alkylthiophenes.

35. A method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 135 degrees Celsius and at most 175 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C2-C3 alkylthiophenes.

36. A method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 139 degrees Celsius and at most 141 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C2 alkylthiophenes.

37. A method of manufacturing a concentrated thiophenic mixture comprising: (i) subjecting an oil comprising between 10% wt/wt and 40% wt/w alkylthiophenes and at least 50% C_{N}H_{M} hydrocarbons to a fractional distillation to recover a fraction having boiling points between at least 161 degrees Celsius and at most 163 degrees Celsius; and (ii) subjecting fluids of the recovered fraction to a cryogenic separation to recover a concentrated thiophenic mixture comprising at least 50% wt/wt or at least 70% wt/wt or at least 90% wt/wt or at least 95% wt/wt or at least 99% wt/wt C3 alkylthiophenes.

38. An oil recovery method comprising:
a. injecting an enhanced oil recovery (EOR) fluid comprising alkylthiophenes into a target subsurface hydrocarbon-containing formation via one or more wells situated therein, a majority of sulfur compounds of the EOR fluid being alkylthiophenes; and
b. recovering, via one or more wells in the target formation, oil and/or bitumen and/or pyrolysis liquids and/or mobilized hydrocarbon liquids that are mobilized by the injected EOR fluid.

39. The method of claim 38 wherein a density of the injected EOR fluid is between 0.95 and 1.05 g/cc.

40. The method of any previous claim wherein the injected EOR fluid comprises primarily alkylthiophenes, or at least 75% wt/wt alkylthiophenes, or at least 90% wt/wt alkylthiophenes, or at least 95% wt/wt alkylthiophenes or at least 99% wt alkylthiophenes.

41. The method of any previous claim wherein an atmospheric boiling point of the EOR fluid is between about 135° C. and about 175° C.

42. The method of any preceding claim wherein a majority, or a substantial majority, of alkylthiophenes of the injected EOR fluid are C1-C3 alkylthiophenes.

43. The method of any preceding claim wherein a majority, or a substantial majority, of alkylthiophenes of the injected EOR fluid are methyl-thiophene or di-methyl-thiophene or tri-methyl-thiophene.

44. The method of any preceding claim wherein a majority, or a substantial majority, of alkylthiophenes of the injected EOR fluid are C2-C3 alkylthiophenes.

45. The method of any preceding claim wherein a majority, or a substantial majority, of alkylthiophenes of the injected EOR fluid are di-methyl-thiophene, or tri-methyl-thiophene.

46. The method of any of claims 38-45 wherein a majority, or a substantial majority, of alkylthiophenes of the injected EOR fluid are C2 alkylthiophenes.

47. The method of any of claims 38-45 wherein a majority, or a substantial majority, of alkylthiophenes of the injected EOR fluid are di-methyl-thiophenes.

48. The method of any of claims 38-45 wherein a majority, or a substantial majority, of alkylthiophenes of the injected EOR fluid are C3 alkylthiophenes.

49. The method of any of claims 38-45 wherein a majority, or a substantial majority, of alkylthiophenes of the injected EOR fluid are tri-methyl-thiophenes.

50. The method of any of claims 38-45 wherein a majority, or a substantial majority, of alkylthiophenes of the injected EOR fluid are methyl-thiophenes.

51. The method of any previous claim wherein the EOR fluid is insoluble in water.

52. The method of any previous claim wherein the hydrocarbon-containing formation is at residual hydrocarbon saturation following waterflood.

53. The method of any previous claim wherein a slug of the EOR fluid comprising alkylthiophenes is followed by brine or brine containing polymer.

54. The method of any preceding claim wherein the hydrocarbon-containing formation is a tar sands formation or a heavy oil formation.

55. The method of any preceding claim wherein the recovered hydrocarbons comprise mobilized bitumen.

56. The method of any previous claim wherein a temperature of the injected EOR fluid is at least 100 degrees Celsius.

57. The method of any previous claim wherein a temperature of the injected EOR fluid is at least 200 degrees Celsius.

58. The method of any preceding claim wherein a majority of the recovered alkylthiophenes are re-injected into the formation or into another subsurface formation.

59. The method of any preceding claim further comprising distilling from the recovered hydrocarbon mixture a majority of the alkylthiophenes to form a second mixture.

60. The method of any preceding claim wherein the a majority of the second mixture is re-injected into target subsurface hydrocarbon-containing formation or injected into a different subsurface hydrocarbon-containing formation.

61. The method of any of claims 59-60 wherein the second mixture has an alkylthiophene concentration that is at most 50% that of the recovered hydrocarbon mixture.

62. The method of any preceding claim where the injecting and the producing is via the same well.

63. The method of any preceding claim where the injecting and the producing is via different wells.

64. The method of any preceding claim wherein within the subsurface formation the EOR fluid acts as a solvent for oil and/or bitumen contained in the formation.

65. The method of any preceding claim wherein within the subsurface formation the EOR fluid boils the in situ brine which steam distills, within the formation, oil and/or bitumen contained in the formation.

66. The method of any preceding claim wherein, when mixed with bitumen of the subsurface formation and within the subsurface formation, the EOR fluid lowers the viscosity of the bitumen by a factor of at least 10, preferably of at least 100.

67. The method of any preceding claim wherein the injected EOR fluid is pre-heated to a temperature of between 50 degrees Celsius and 200 degrees Celsius.

68. The method of any preceding claim wherein, when mixed with bitumen of the subsurface formation and within the subsurface formation, at a temperature of between 50 degrees Celsius and 200 degrees Celsius, the EOR fluid lowers the viscosity of the bitumen by a factor of at least 100, and preferably by at least 1000.

69. The method of any preceding claim, plus distilling the EOR fluid from the recovered oil and/or bitumen, re-injecting the EOR fluid into the target formation for additional recovery of oil and/or bitumen.

70. The method of any previous claim wherein the EOR fluid is at least 10% wt/wt or at least 15% wt/wt or at least 20% wt/wt sulfur.

71. The method of any previous claim wherein an atmospheric boiling point of the EOR fluid is between about 80° C. and about 175° C.

72. The method of claim 71 wherein an atmospheric boiling point of the EOR fluid is in one of the ranges: (i) between about 113 degrees Celsius and about 119 degrees Celsius; (ii) between about 137 degrees Celsius and about 143 degrees Celsius; and (iii) between about 159 degrees Celsius and about 165 degrees Celsius.

73. The method of claim 72 wherein the atmospheric boiling point of the EOR fluid is at least 100° C. or at least 110° C.

74. The method of any preceding claim wherein a majority, or a substantial majority, of alkylthiophenes of the injected EOR fluid are thiophene C₄H₄S or C1-C4 alkylthiophenes.

75. The method of any preceding claim wherein the target formation is a kerogenous chalk.

76. A method of producing a thiophenic fluid mixture, the method comprising:
a. pyrolyzing type IIs kerogen to generate condensable pyrolysis fluids therefrom; and
b. forming from the pyrolysis liquids a thiophenic fluid mixture comprising at least 50% wt/wt alkylthiophenes.

77. The composition of any claim 76 wherein the thiophenic fluid mixture formed from the pyrolysis liquids comprises at least 50% wt/wt or at least 70% wt/wt or at least 95% wt/wt or at least 99% wt/wt methylthiophenes.

78. The composition of any claim 76 wherein the thiophenic fluid mixture formed from the pyrolysis liquids comprises at least 50% wt/wt or at least 70% wt/wt or at least 95% wt/wt or at least 99% wt/wt dimethylthiophenes.

79. The composition of any claim 76 wherein the thiophenic fluid mixture formed from the pyrolysis liquids comprises at least 50% wt/wt or at least 70% wt/wt or at least 95% wt/wt or at least 99% wt/wt trimethylthiophenes.

80. The method of any of claims 76-79 wherein the pyrolyzing is performed in situ.

81. The method of any of claims 76-80 wherein the pyrolysis occurs primarily at temperatures below 290 degrees Celsius.

82. The method of any of claims 76-81 wherein the forming includes (i) subjecting the pyrolysis liquids or a derivative thereof to a fractional distillation to recover a fraction having boiling points between at least 135 degrees Celsius and at most 175 degrees Celsius; and (ii) subjecting fluids of the recovered fraction to an extractive distillation with a polar organic solvent having a boiling point of at least 180 degrees Celsius.

83. The method of any of claims 76-82 wherein the thiophenic fluid mixture comprises at least 75% wt/wt alkylthiophenes, or at least 90% wt/wt alkylthiophenes, or at least 95% wt/wt alkylthiophenes or at least 99% wt alkylthiophenes.

84. The method of any of claims 76-83 wherein the thiophenic fluid mixture comprises at least 75% wt/wt methyl-thiophenes, or at least 90% wt/wt methyl-thiophenes, or at least 95% wt/wt methyl-thiophenes or at least 99% methyl-thiophenes.

85. The method of any of claims 76-84 wherein the thiophenic fluid mixture comprises at least 5% wt/wt or at least 10% wt/wt or at least 20% wt/wt hydrocarbons C_{N}H_{M} hydrocarbons wherein N and M are both positive integers, and a value of N is between 5 and 12.

86. The method of any of claims 76-85 wherein (i) the forming includes subjecting the condensable pyrolysis fluids or a derivative thereof to a distillation process to recover fluids having an atmospheric boiling point in the 75° C.-175° C. range and (ii) the thiophenic fluid mixture is derived from the 75° C.-175° C. range fluids recovered by the distillation.

87. The method of any of claims 76-85 wherein (i) the forming includes subjecting the condensable pyrolysis fluids or a derivative thereof to a distillation process to recover fluids having an atmospheric boiling point in the 135° C.-175° C. range and (ii) the thiophenic fluid mixture is derived from the 135° C.-175° C. range fluids recovered by the distillation.

88. The method of any of claims 76-85 wherein the forming includes subjecting the condensable pyrolysis fluids to a chemical extraction process by a polar organic solvent having a boiling point above 160 degrees Celsius or above 180 degrees Celsius.

89. The method of any of claims 76-85 wherein the forming includes subjecting the condensable pyrolysis fluids to a chemical extraction process by a polar organic solvent which differentiates between alkylthiophenes and C_{N}H_{M} hydrocarbons wherein N and M are both positive integers, and a value of N is between 5 and 12.

90. The method of any of claims 76-89 wherein the forming includes subjecting the condensable pyrolysis fluids to a cryogenic separation process.

91. The method of any of claims 76-90 wherein a majority, or a substantial majority, of alkylthiophenes of the thiophenic fluid mixture are C1-C3 alkylthiophenes.

92. The method of any of claims 76-90 wherein a majority, or a substantial majority, of alkylthiophenes of the thiophenic fluid mixture are methyl-thiophene or di-methyl-thiophenes or tri-methyl-thiophenes.

93. The method of any of claims 76-90 wherein a majority, or a substantial majority, of alkylthiophenes of the thiophenic fluid mixture are C2-C3 alkylthiophenes.

94. The method of any of claims 76-90 wherein a majority, or a substantial majority, of alkylthiophenes of the thiophenic fluid mixture are di-methyl-thiophenes or tri-methyl thiopheness.

95. The method of any of claims 76-94 wherein the pyrolysis is carried out within a pit or an impoundment.

96. An EOR fluid generated by a method of any of claims 76-95.

97. User of the composition of any previous claim as an EOR fluid.
